(19) United States
(12) Patent Application Publication
(10) Pub. No.: US 2026/0130655 A1
(43) Pub. Date: May 14, 2026

(54) TISSUE SAMPLE HOLDER DRIVE MECHANISMS FOR BIOPSY DEVICE

(71) Applicant: Devicor Medical Products, Inc., Cincinnati, OH (US)

(72) Inventors: Eric J. MILLER, Cincinnati, OH (US); Jack A. RANDALL, Cincinnati, OH (US); Garrett A. HOUSEHOLDER, Cincinnati, OH (US); James O. ROGERS, III, Milford, OH (US); David R. SANDER, Lebanon, OH (US); Spencer CHAMBERLAIN, Cincinnati, OH (US); Justin REBELLINO, Cincinnati, OH (US); Andrew SMALL, Cincinnati, OH (US); David C. McBREEN, West Chester, OH (US); Lyle JAMES, Cincinnati, OH (US)

(21) Appl. No.: 19/438,913

(22) Filed: Jan. 2, 2026

Related U.S. Application Data
(63) Continuation of application No. PCT/US24/34508, filed on Jun. 18, 2024.
(60) Provisional application No. 63/525,430, filed on Jul. 7, 2023.

(51) Int. Cl. A61B 10/02

(52) U.S. Cl. CPC A61B 10/0275; A61B 2010/0208

(57) ABSTRACT

A biopsy device includes a body, a needle, a hollow cutter, a tissue sample holder, and a drive mechanism. The needle extends distally from the body and includes a lateral aperture. The cutter is movable relative to the needle to sever a tissue sample and defines a cutter lumen. The tissue sample holder includes a rotatable member and one or more tissue receiving trays. The rotatable member is configured to receive each tissue receiving tray of the one or more tissue receiving trays. The rotatable member is further configured to rotate relative to the cutter to index each tissue receiving tray relative to the cutter. The drive mechanism includes a drive screw. The drive screw is configured to drive both rotation and translation of the rotatable member.

### PRIORITY

This application claims priority to U.S. Provisional Application Ser. No. 63/525,430, entitled “Tissue Sample Holder Drive Mechanisms for Biopsy Device,” filed on Jul. 7, 2023, the disclosure of which is incorporated by reference herein.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; and U.S. Pat. No. 8,118,755, entitled “Biopsy Sample Storage,” issued Feb. 21, 2012. The disclosure of each of the above-cited U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006; U.S. Pat. Pub. No. 2008/0146962, entitled “Biopsy System with Vacuum Control Module,” published Jun. 19, 2008; U.S. Pat. Pub. No. 2008/0214955, entitled “Presentation of Biopsy Sample by Biopsy Device,” published Sep. 4, 2008; U.S. Pat. Pub. No. 2008/0221480, entitled “Biopsy Sample Storage,” published Sep. 11, 2008; U.S. Pat. Pub. No. 2009/0131821, entitled “Graphical User Interface For Biopsy System Control Module,” published May 21, 2009; U.S. Pat. Pub. No. 2009/0131820, entitled “Icon-Based User Interface on Biopsy System Control Module,” published May 21, 2009; U.S. Pat. Pub. No. 2009/0216152, entitled “Needle Tip for Biopsy Device,” published Aug. 27, 2009; U.S. Pat. Pub. No. 2010/0113973, entitled “Biopsy Device with Rotatable Tissue Sample Holder,” published May 6, 2010; U.S. Pat. Pub. No. 2010/0152610, entitled “Hand Actuated Tetherless Biopsy Device with Pistol Grip,” published Jun. 17, 2010; U.S. Pat. Pub. No. 2010/0160819, entitled “Biopsy Device with Central Thumbwheel,” published Jun. 24, 2010; U.S. Pat. Pub. No. 2010/0160824, entitled “Biopsy Device with Discrete Tissue Chambers,” published Jun. 24, 2010; U.S. Pat. Pub. No. 2010/0317997, entitled “Tetherless Biopsy Device with Reusable Portion,” published Dec. 16, 2010; U.S. Pat. Pub. No. 2012/0109007, entitled “Handheld Biopsy Device with Needle Firing,” published May 3, 2012; U.S. Pat. Pub. No. 2012/0265095, entitled “Biopsy Device with Motorized Needle Firing,” published Oct. 18, 2012; U.S. Pat. Pub. No. 2012/0283563, entitled “Biopsy Device with Manifold Alignment Feature and Tissue Sensor,” published Nov. 8, 2012; U.S. Pat. Pub. No. 2012/0310110, entitled “Needle Assembly and Blade Assembly for Biopsy Device,” published Dec. 6, 2012; U.S. Pat. Pub. No. 2013/0041256, entitled “Access Chamber and Markers for Biopsy Device,” published Feb. 14, 2013; U.S. Pat. Pub. No. 2013/0053724, entitled “Biopsy Device Tissue Sample Holder with Bulk Chamber and Pathology Chamber,” published Feb. 28, 2013; U.S. Pub. No. 2013/0144188, entitled “Biopsy Device With Slide-In Probe,” published Jun. 6, 2013; U.S. Pub. No. 2013/0324882, entitled “Control for Biopsy Device,” published Dec. 5, 2013; U.S. Pub. No. 2013/0218047, entitled “Biopsy Device Valve Assembly,” published Aug. 22, 2013; and U.S. Pub. No. 2014/0039343, entitled “Biopsy System,” published Feb. 6, 2014. The disclosure of each of the above-cited U.S. Patent Application Publications, U.S. Non-Provisional Patent Applications, and U.S. Provisional Patent Applications is incorporated by reference herein.

Some biopsy devices may include a tissue sample holder to receive one or more tissue samples acquired by the biopsy device. In some examples, one or more portions of such a tissue sample holder may be rotatable or otherwise movable relative to a portion of the biopsy device to receive received tissue samples in separate portions of the tissue sample holder. An aspect of such relative movement is sealing between portions of the tissue sample holder. In some circumstances, such sealing may introduce certain operational challenges such as increasing the force used for movement, wear on sealing structures, or inconsistent sealing. Thus, it may be desirable to include certain features in a biopsy device to promote sealing between the biopsy device and a tissue sample holder, while permitting relative movement between a portion of the tissue sample holder and a portion of the biopsy device.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a schematic view of an exemplary biopsy system;

FIG. 2 depicts a perspective view of an exemplary probe for use in the biopsy system of FIG. 1;

FIG. 3 depicts a partial perspective exploded view of the probe of FIG. 2;

FIG. 4 depicts a perspective exploded view of a tissue sample holder of the probe of FIG. 2;

FIG. 5 depicts a schematic view of a tissue sample holder drive assembly for use with the tissue sample holder of FIG. 4;

FIG. 6 depicts a perspective view of another tissue sample holder drive assembly that may be incorporated into the tissue sample holder of FIG. 4;

FIG. 7 depicts a perspective exploded view of the tissue sample holder drive assembly of FIG. 6;

FIG. 8 depicts a detailed perspective view of a threaded receiving feature of the tissue sample holder drive assembly of FIG. 6;

FIG. 9 depicts a perspective view of a hard stop feature of the tissue sample holder drive assembly of FIG. 6;

FIG. 10A depicts a perspective view of the tissue sample holder drive assembly of FIG. 6, the tissue sample holder drive assembly being manipulated to translate a portion of the tissue sample holder of FIG. 4;

FIG. 10B depicts another perspective view of the tissue sample holder drive assembly of FIG. 6, the tissue sample holder drive assembly being manipulated to translate and rotate a portion of the tissue sample holder of FIG. 4;

FIG. 11 depicts a detailed perspective view of the tissue sample holder drive assembly of FIG. 6, the hard stop feature of FIG. 9 engaging a corresponding hard stop of a threaded driver;

FIG. 12 depicts a perspective view of yet another tissue sample holder drive assembly that may be incorporated into the tissue sample holder of FIG. 4;

FIG. 13 depicts an exploded perspective view of the tissue sample holder drive assembly of FIG. 12;

FIG. 14 depicts a detailed perspective view of a threaded receiving feature of the tissue sample holder drive assembly of FIG. 12;

FIG. 15 depicts a detailed elevational view of the threaded receiving feature of FIG. 14;

FIG. 16A depicts another perspective view of the tissue sample holder drive assembly of FIG. 12, the tissue sample holder drive assembly being manipulated to translate a portion of the tissue sample holder of FIG. 4 distally;

FIG. 16B depicts yet another perspective view of the tissue sample holder drive assembly of FIG. 12, the tissue sample holder drive assembly being manipulated to translate a portion of the tissue sample holder of FIG. 4 proximally;

FIG. 17A depicts a detailed front elevational view of the tissue sample holder drive assembly, the threaded receiving feature engaging a rotation lock to prevent rotation of a portion of the tissue sample holder of FIG. 4;

FIG. 17B depicts another detailed front elevational view of the tissue sample holder drive assembly, the threaded receiving feature disengaging the rotation lock of FIG. 17A to permit rotation of a portion of the tissue sample holder of FIG. 4;

FIG. 18 depicts a perspective view of an exemplary alternative tissue sample holder that may be readily incorporated into the probe of FIG. 2;

FIG. 19 depicts a side cutaway view of the probe of FIG. 2 with the tissue sample holder of FIG. 18 incorporated therein;

FIG. 20 depicts a side exploded elevational view of another exemplary alternative tissue sample holder that may be readily incorporated into the probe of FIG. 2;

FIG. 21 depicts a front cross-sectional view of the tissue sample holder of FIG. 20, a tissue receiving tray of the tissue sample holder being received within a receiving barrel of the tissue sample holder;

FIG. 22 depicts a side elevational view of the tissue sample holder of FIG. 20 incorporated into the probe of FIG. 2, the tissue sample holder in an initial disassembled state;

FIG. 23 depicts another side elevational view of the tissue sample holder of FIG. 20 incorporated into the probe of FIG. 2, the tissue sample holder in an assembled state and being manipulated to seal relative to the probe;

FIG. 24 depicts a side exploded elevational view of yet another exemplary alternative tissue sample holder that may be readily incorporated into the probe of FIG. 2;

FIG. 25 depicts a side elevational view of the tissue sample holder of FIG. 24 incorporated into the probe of FIG. 2, the tissue sample holder in an initial disassembled state;

FIG. 26 depicts another side elevational view of the tissue sample holder of FIG. 24 incorporated into the probe of FIG. 2, the tissue sample holder in an assembled state and being manipulated to seal relative to the probe; and

FIG. 27 depicts a perspective view of an exemplary alternative seal that may be readily incorporated into the probe of FIG. 2.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Overview of Exemplary Biopsy System

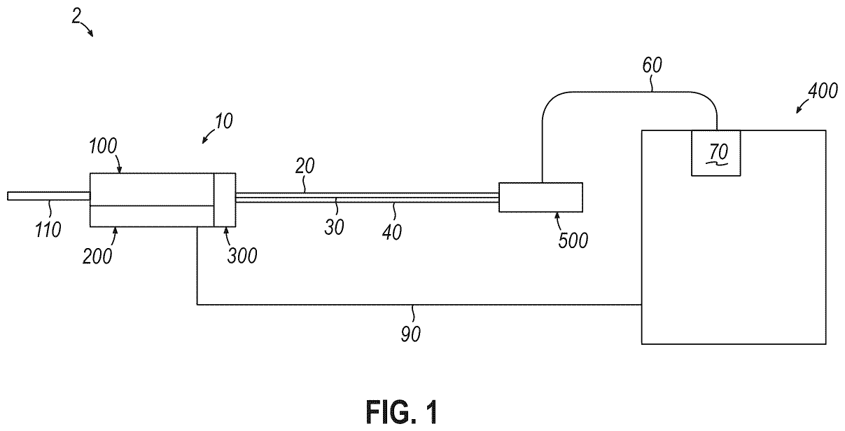

FIG. 1 depicts an exemplary biopsy system (2) comprising a biopsy device (10) and a vacuum control module (400). Biopsy device (10) of this example comprises a probe (100) and a holster (200). A needle (110) extends distally from probe (100), and is configured for insertion into a patient's tissue to obtain tissue samples. These tissue samples are deposited in a tissue sample holder (300) at the proximal end of probe (100), as will also be described in greater detail below. It should also be understood that the use of the term “holster” herein should not be read as requiring any portion of probe (100) to be inserted into any portion of holster (200). In some examples probe (100), holster (200), and or both may include tabs, prongs, or other features to facilitate coupling of probe (100) to holster (200). Of course, a variety of other types of structures, components, features, etc. (e.g., bayonet mounts, latches, clamps, clips, snap fittings, etc.) may be used to provide removable coupling of probe (100) and holster (200). Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Biopsy device (10) of the present example is configured to mount to a table or fixture, and be used under stereotactic guidance. Of course, biopsy device (10) may instead be used under ultrasound guidance, MRI guidance, PEM guidance, BSGI guidance, or otherwise. It should also be understood that biopsy device (10) may be sized and configured such that biopsy device (10) may be operated by a single hand of a user. In particular, a user may grasp biopsy device (10), insert needle (110) into a patient's breast, and collect one or a plurality of tissue samples from within the patient's breast, all with just using a single hand. Alternatively, a user may grasp biopsy device (10) with more than one hand and/or with any desired assistance. In some settings, the user may capture a plurality of tissue samples with just a single insertion of needle (110) into the patient's breast. Such tissue samples may be pneumatically deposited in tissue sample holder (300), and later retrieved from tissue sample holder (300) for analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various exemplary components, features, configurations, and operabilities of biopsy device (10) will be described in greater detail below; while other suitable components, features, configurations, and operabilities will be apparent to those of ordinary skill in the art in view of the teachings herein.

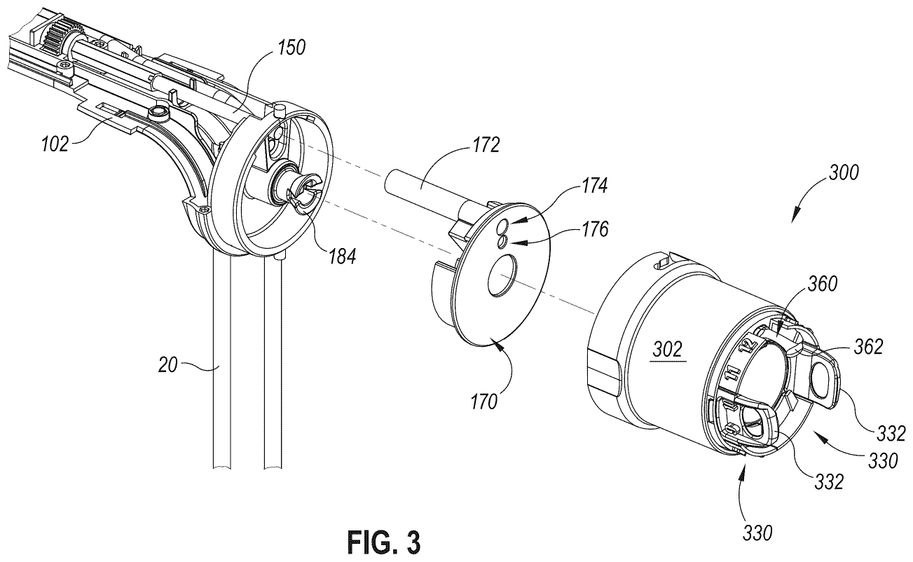

Holster (200) of the present example includes one or more gears (not shown) and one or more motors (not shown) to drive such gears to further drive one or more functions of probe (100). Such gears may be exposed through an upper surface of holster (200), and mesh with corresponding gears (not shown) of probe (100) when probe (100) and holster (200) are coupled together. Such gears may be configured to drive actuation of various components of probe (100) such as a cutter (150) (see FIG. 3) disposed within needle (110), rotation of needle (110), and/or movement of one or more components of tissue sample holder (300). By way of example only, such drive mechanisms may be constructed in accordance with the teachings of U.S. Pub. No. 2008/0214955, the disclosure of which is incorporated by reference herein. As another merely illustrative example, such drive mechanisms may be constructed in accordance with the teachings of U.S. Pub. No. 2010/0160819, the disclosure of which is incorporated by reference herein.

As noted above, holster (200) may include one or more motors to facilitate movement of one or more components of probe. All motors referred to herein are contained within holster (200) in the present example and receive power from vacuum control module (400) via cable (90). In addition, data may be communicated between vacuum control module (400) and holster (200) via cable (90). In some other versions, one or more motors are powered by one or more batteries located within holster (200) and/or probe (100). It should therefore be understood that, as with other components described herein, cable (90) is merely optional. As yet another merely illustrative variation, motors may be powered pneumatically, such that cable (90) may be substituted with a conduit communicating a pressurized fluid medium to holster (200). As still other merely illustrative variation, cable (90) may include one or more rotary drive cables that are driven by motors that are located external to holster (200). It should also be understood that two or three of the motors may be combined as a single motor. Other suitable ways in which various the motors may be driven will be apparent to those of ordinary skill in the art in view of the teachings herein.

### II. Exemplary Probe

Probe (100) of the present example includes a needle (110) extending distally from a body (102) of probe (100) that is inserted into a patient's tissue to obtain tissue samples. These tissue samples are deposited in a tissue sample holder (300) at the proximal end of probe (100). As shown in FIG. 1, vacuum control module (400) is coupled with probe (100) via a valve assembly (500) and tubes (20, 30, 40, 60), which is operable to selectively provide vacuum, saline, atmospheric air, and venting to probe (100). Vacuum or other fluid mediums may be provided by vacuum control module (500) via a vacuum canister (70) in communication with valve assembly (500) via tube (60). The internal components of the valve assembly of the present example are configured and arranged as described in U.S. Pub. No. 2013/0218047, entitled “Biopsy Device Valve Assembly,” published Aug. 22, 2013, the disclosure of which is incorporated by reference herein.

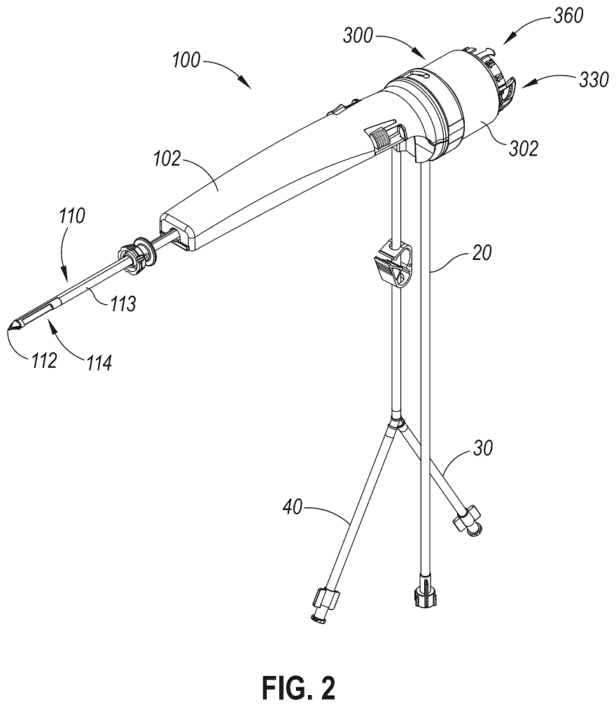

As best seen in FIG. 2, needle (110) of the present example comprises a cannula (113) having a tissue piercing tip (112), a lateral aperture (114) located proximal to tip (112). Tissue piercing tip (112) is configured to pierce and penetrate tissue, without requiring a high amount of force, and without requiring an opening to be pre-formed in the tissue prior to insertion of tip (112). Alternatively, tip (112) may be blunt (e.g., rounded, flat, etc.) if desired. By way of example only, tip (112) may be configured in accordance with any of the teachings in U.S. Pat. No. 8,801,742, entitled “Needle Assembly and Blade Assembly for Biopsy Device,” issued Aug. 12, 2014, the disclosure of which is incorporated by reference herein. As another merely illustrative example, tip (112) may be configured in accordance with at least some of the teachings in U.S. Pub. No. 2013/0144188, entitled “Biopsy Device with Slide-In Probe,” filed Published 6, 2013, the disclosure of which is incorporated by reference herein. Other suitable configurations that may be used for tip (112) will be apparent to those of ordinary skill in the art in view of the teachings herein.

Lateral aperture (114) is sized to receive prolapsed tissue during operation of device (10). A hollow tubular cutter (150) (see FIG. 3) having a sharp distal edge is located within needle (110) and extends proximally through body (102) to tissue sample holder (300). Cutter (150) is operable to rotate and translate relative to needle (110) and past lateral aperture (114) to sever a tissue sample from tissue protruding through lateral aperture (114). For instance, the cutter may be moved from an extended position to a retracted position, thereby “opening” lateral aperture (114) to allow tissue to prolapse therethrough; then from the retracted position back to the extended position to sever the prolapsed tissue. As will be described in greater detail below, needle (110) may be rotated to orient lateral aperture (114) at any desired angular position about the longitudinal axis of needle (110).

In some examples, needle (110) may be constructed in accordance with one or more teachings of U.S. Pat. No. 7,918,803, entitled “Methods and Devices for Automated Biopsy and Collection of Soft Tissue,” issued Apr. 5, 2011, the disclosure of which is incorporated by reference herein. In yet other examples, needle may be construed in accordance with one or more teachings of U.S. Pub. No. 2007/0032742, entitled “Biopsy Device with Vacuum Assisted Bleeding Control,” published Feb. 8, 2007, the disclosure of which is incorporated by reference herein. Of course, as with any other component described herein, any other suitable configurations may be used.

Although not shown, it should be understood that body (102) of probe (100) may include a variety of components to facilitate operational functionality of probe (100). By way of example only, such components may be configured to drive components such as cutter (150), rotation of needle (110), and/or movement of one or more portions of tissue sample holder (300). In some examples, such components are further configured in accordance with at least some of the teachings of U.S. Pat. Pub. No. 2008/0214955, the disclosure of which is incorporated by reference herein.

As best seen in FIG. 3, tissue sample holder (300) of the present example provides a plurality of discrete chambers that are configured to receive tissue samples that are severed by cutter (150) disposed within needle (110) and communicated proximally through a lumen (not shown) defined by cutter (150). In particular, and as will be described in greater detail below, tissue sample holder (300) includes one or more tissue receiving trays (330) that are removably engaged with a manifold (310) (also referred to as a rotatable member). Manifold (310) is removably engaged with a grasping feature (184) associated with a rotation member (not shown) disposed within body (102) of probe (100). The rotation member is longitudinally fixed relative to body (102) yet is rotatable relative to body (102). In some examples, the rotation member includes an integral gear or other drive feature, which is configured to engage a portion of holster (200) to permit rotation of manifold (310) via the one or more motors disposed within holster (200). Thus, one or more gears of probe (100) and one or more gears of holster (200) cooperate to rotate manifold (310) to index tissue chambers relative to the lumen of cutter (150) as will be described in greater detail below. A transparent cover (302) is positioned about manifold (310) and is removably secured to body (101). While bayonet features provide coupling between cover (302) and body (102), it should be understood that any suitable type of coupling may be used. Manifold (310) is freely rotatable within cover (302). However, manifold (310) is engaged with cover (302) such that manifold (310) will decouple relative to body (102) when cover (302) is removed from body (101). In other words, manifold (310) may be selectively coupled with and removed relative to body (101) by coupling and removing cover (302) from body (102).

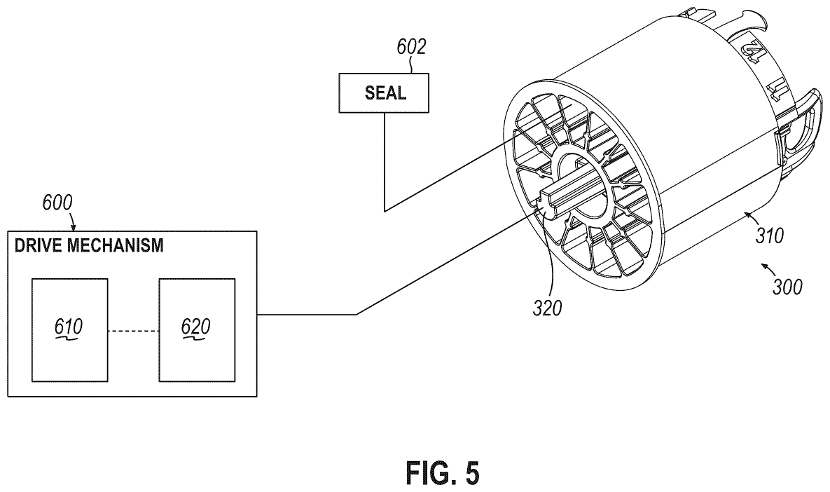

As best seen in FIGS. 2 and 3, manifold (310) of the present example defines a plurality of chambers in the form of passages (312) that extend longitudinally through manifold (310) and that are angularly arrayed about the central axis of manifold (310). As will be described in greater detail below, passages (312) receive trays (330). An additional passage (313) is associated with a plug (360), as will also be described in greater detail below. Manifold (310) also includes a central shaft (320) (see FIG. 5), which is configured to removably engage grasping feature (184). Central shaft (320) is configured to couple with grasping feature (184) upon coupling of cover (302) with body (102), as described above. Engagement between central shaft (320) and grasping feature (184) provides rotation of manifold (310) upon rotation of the rotation member associated with gasping feature (184).

As best seen in FIG. 3, a sealing member (170) is provided at the proximal end of body (102) and interfaces with the distal face of manifold (310). In the present example, sealing member (170) comprises rubber, though it should be understood that any other suitable material(s) may be used. Sealing member (170) includes a longitudinally extending cutter seal (172), which receives cutter (150) and seals against the exterior of cutter (150). The proximal end of cutter (150) remains within cutter seal (172) throughout the full range of travel of cutter (150). Cutter seal (172) maintains a fluid tight seal against cutter (150) during this full range of motion, including during rotation and translation of cutter (150). An opening (174) is positioned at the proximal end of cutter seal (170). This opening (174) is configured to align with whichever passage (312, 313) is at the 12 o'clock position. Another opening (176) is positioned below opening (174). Opening (176) is configured to align with manifold (310) to provide vacuum to whichever passage (312, 313) is at the 12 o'clock position. It should be understood that sealing member (170) and manifold (310) are generally configured to cooperatively provide fluid communication between tube (20) and the lumen of cutter (150) via whichever passage (312, 313) is at the 12 o'clock position. It should be understood that sealing member (170) of the present example maintains a fluid tight seal against the distal face of manifold (310), even as manifold (310) is rotated relative to sealing member (170).

In the present example, sealing member (170) is configured to correspond to the distal end of manifold (310). In other words, sealing member (170) defines a generally circular shape that is configured to cover the entire distal end of manifold (310), thereby sealing all passages (312, 313) simultaneously. In other versions, sealing member (170) is configured to seal only a portion of manifold (310). As described above, only the particular passage (312, 313) in the 12 o'clock position is actively subjected to vacuum or other fluid media during use of biopsy device (10). Thus, in some examples, it may be desirable to only seal the particular passage (312, 313) in the 12 o'clock position and optionally one or more adjacent passages. In such examples, sealing member (170) may be triangular or wedge-shaped. In other examples, sealing member (170) is omitted and dedicated sealing structures (e.g., o-rings) may be used in lieu-of sealing member (170). Specific dedicated sealing structures are described in greater detail below and may be readily incorporated into probe (100) of the present example.

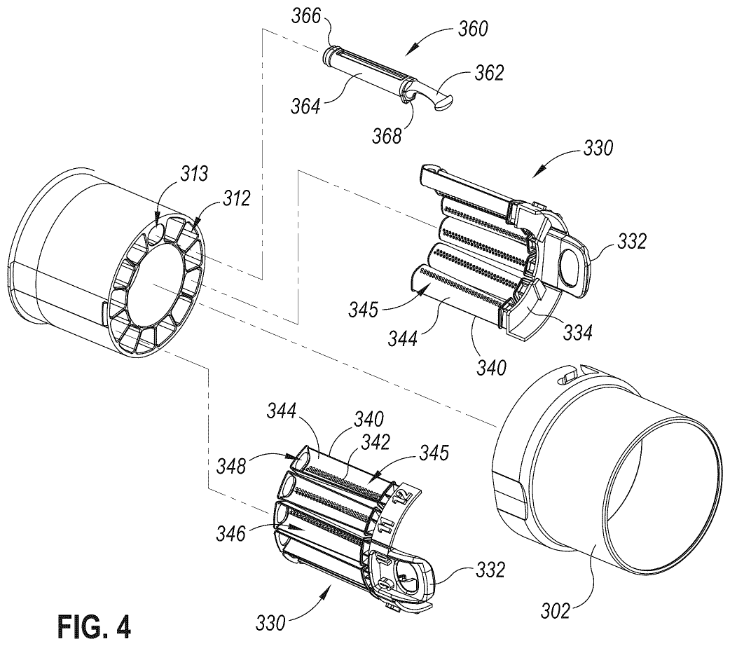

As noted above, tissue receiving trays (330) are configured to removably engage manifold (310). As best seen in FIG. 4, each tissue receiving tray (330) of the present example includes a grip (332), a proximal wall (334), and a plurality of strips (340) extending distally from proximal wall (334). Strips (340) are sized and configured for insertion into associated passages (312) of manifold (310). Each strip (340) includes a pair of sidewalls (344) and a floor (342). Each pair of sidewalls (344) and floor (342) together define a corresponding tissue sample chamber (346). An opening (348) is provided at the distal end of each tissue sample chamber (346). Opening (348) is sized and positioned to correspond with opening (174) of sealing member (170). Thus, the lumen of cutter (150) is in fluid communication with the tissue sample chamber (346) of the strip (340) inserted in the passage (312) that is at the 12 o'clock position.

Each floor (342) includes a plurality of openings (345) that provide fluid communication between tissue sample chamber (346) of strip (340) and a lateral recess or gap defined by a given passage (312) beneath strip (340). Thus, vacuum, atmospheric air, etc. that is communicated to opening (176) via tube (20) is further communicated to the lumen of cutter (150) via the lateral recess or gap, openings (345), and tissue sample chamber (346). During operation of biopsy device (10), tissue samples severed by the distal edge of cutter (150) are communicated proximally through the lumen of cutter (150) and are then deposited into the tissue sample chamber (346) that is aligned with cutter (150). Manifold (310) is rotated to successively align tissue sample chambers (346) with cutter (150), enabling several tissue samples to be separately deposited in different tissue sample chambers (346) during operation of biopsy device (10). Bodily fluids and saline, etc. that are pulled through lumen (151) will pass through tissue sample holder (300) and tube (20) and are eventually deposited in vacuum canister (70).

It should be understood that manifold (310) and/or trays (330) may be configured in numerous other ways. By way of example only, manifold (310) and/or trays (330) may be configured in accordance with at least some of the teachings of U.S. Pat. Pub. No. 2008/0214955, the disclosure of which is incorporated by reference herein. As another merely illustrative example, manifold (310) and/or trays (330) may be configured in accordance with at least some of the teachings of U.S. Pat. Pub. No. 2010/0160824, the disclosure of which is incorporated by reference herein. It should also be understood that tissue sample holder (300) need not necessarily position chambers (346) coaxially with the lumen of cutter (150). Tissue sample holder (300) may index chambers (346) relative to cutter (150) in any other suitable fashion. For instance, chambers (346) may extend along axes that are always offset from the axis of cutter (150), along axes that are oblique or perpendicular relative to the axis of cutter (150), or along other axes. Similarly, it should be understood that manifold (310) may rotate about an axis that is oblique or perpendicular relative to the axis of cutter (150). Yet in other examples, tissue sample holder trays (330) may be used in conjunction with an imaging system which may be configured in accordance with at least some of the teachings of U.S. App. No. Ser. No. 14/208,354, entitled “Biopsy Device,” filed Sep. 18, 2014, the disclosure of which is incorporated by reference herein. Still other suitable configurations will be apparent to those of ordinary skill in the art in view of the teachings herein.

As best seen in FIG. 4 and as noted above, tissue sample holder (300) of the present example includes a plug (360) that is received in a dedicated passage (313) of manifold (310). Plug (360) includes a grip (362) and a longitudinally extending body (364). Body (364) extends through part of the length of passage (313). Plug (360) includes a pair of seals (366, 368) that seal against the interior of passage (313) when plug (360) is fully inserted in passage (313). Seals (366, 368) thus keep passage (313) fluid tight when plug (360) is inserted in passage (313). Passage (313) is configured to receive a shaft of a biopsy site marker applier or other device. Passage (313) may also receive an instrument for delivering medicine, etc. to a biopsy site. By way of example only, passage (313) may receive an adapter configured to provide an interface between passage (313) and a conventional medicine delivery device. An example of such an adapter and other uses/configurations for a passage like passage (313) are described in U.S. Pat. Pub. No. 2008/0221480, the disclosure of which is incorporated by reference herein. Plug (360) and/or passage (313) may also be configured and operable in accordance with at least some of the teachings of U.S. Pat. No. 8,938,285, entitled “Access Chamber and Markers for Biopsy Device,” issued Jan. 20, 2015, the disclosure of which is incorporated by reference herein. Still other suitable configurations will be apparent to those of ordinary skill in the art in view of the teachings herein. In some other versions, plug (360) and/or passage (313) are simply omitted.

### II. Exemplary Tissue Sample Holder Drive With Sealing Function

In some probes similar to probe (100) described above, it may be desirable to drive movement of one or more portions of a tissue sample holder similar to tissue sample holder (300) described above in one or more particular ways. For instance, as described above, some tissue sample holders include structures such as a manifold similar to manifold (310), which engage a seal or other structure to fluidly isolate chambers or passages of the tissue sample holder relative to the exterior of the tissue sample holder. Additionally, it may be desirable to rotate the manifold to index such chambers or passages with a portion of the probe such as a cutter. In some circumstances, such rotation may have challenges due to friction between the manifold and the seal. For instance, the presence of friction may increase the force used to rotate the manifold. Additionally, the friction may increase wear, contributing to decreased service life of the tissue sample holder itself and/or parts associated with the tissue sample holder such as gears, motors, transmissions, and/or etc. Thus, it may be desirable in some circumstances to move one or more portions of the tissue sample holder in ways that may reduce friction between one or more components of the tissue sample holder and seals or other structures associated with the tissue sample holder.

### A. Exemplary Tissue Sample Holder Drive Components

FIG. 5 shows an exemplary drive mechanism (600) (also referred to as a tissue sample holder drive) that may be readily incorporated into probe (100) described above to facilitate movement of tissue sample holder (300). In the present example, drive mechanism (600) is generally configured to drive a portion of tissue sample holder (300) with both rotational motion and at least some translational motion relative to a seal (602), which may be substantially similar to sealing member (170) described above. As will be described in greater detail below, such rotational and translational motions may facilitate indexing of tissue sample holder (300) relative to cutter (150), while also reducing friction between seal (602) and one or more portions of tissue sample holder (300).

Drive mechanism (600) includes a rotation drive (610) and a translation drive (620). Both rotation drive (610) and translation drive (620) are in communication with manifold (310) of tissue sample holder (300) to drive movement of manifold (310). Although rotation drive (610) and translation drive (620) are shown separately in the present example, it should be understood that in other examples, rotation drive (610) and translation drive (620) are incorporated into a single drive with a single communication pathway to tissue sample holder (300). Alternatively, in other examples, rotation drive (610) and translation drive (620) are entirely separate mechanisms with dedicated communication pathways to tissue sample holder (300). In addition or in the alternative, in some examples, rotation drive (610) and translation drive (620) may be separate, but in communication with each other to facilitate coordination of movements between rotation drive (610) and translation drive (620). Additionally, as will be described in greater detail below, in some examples one or more components of rotation drive (610) and/or translation drive (620) are readily incorporated into one or more components of tissue sample holder (300) such as manifold (310).

Rotation drive (610) is generally configured to drive rotation of manifold (310). Thus, rotation drive (610) is in communication with manifold (310) via central shaft (320) to communicate rotatory power to manifold (310). By way of example only, rotation drive (610) includes a variety of components to facilitate rotation of manifold (310) such as one or more shafts, gears, transmissions, motors, and/or etc.

Translation drive (620) is generally configured to drive translation of manifold (310) proximally and distally. In some examples, such translation is sequential and is coordinated with rotation driven by rotation drive (610). In other examples, such translation is entirely selective and independent of rotation driven by rotation drive (610). As will be described in greater detail below, the particular magnitude of translation driven by translation drive (620) is an amount sufficient to reduce friction at an interface between manifold (310) and seal (602). In some examples, the magnitude of translation driven by translation drive (620) corresponds to 1 to 4 mm. In other examples, other suitable magnitudes of translation may be used as will be apparent to those of ordinary skill in the art in view of the teachings herein.

Translation drive (620) includes a variety of components to facilitate translation of one or more portions of tissue sample holder (300). By way of example only, such components may include cam-based mechanisms, screw-based mechanisms, ratchets, lock collars, gears, shafts, motors and/or etc. As noted above and will be described in greater detail below, in some examples, one or more components of translation drive (620) are integrated with one or more components of rotation drive (610) such that both rotation and translation of manifold (310) is provided by a single mechanism. In other examples, components of translation drive (620) are entirely separate from rotation drive (610) such that rotation and translation of manifold (310) is provided by discrete mechanisms. Although various specific mechanisms are described herein to facilitate rotation and translation of manifold (310), it should be understood that various alternative mechanisms may be used, which may combine one or more components of the mechanisms described herein.

### B. Exemplary Tissue Sample Holder Drive Assembly With Manifold Integration

As noted above, in some examples, it may be desirable to drive rotation and translation of a structure such as manifold (310) using a single drive mechanism. Such a single drive mechanism configuration may be desirable to provide enhanced functionality with a single drive input. For instance, in such configurations, a single motor is used to provide both rotation and translation of manifold (310) or similar structures. Additionally, it may also be desirable to incorporate one or more components of the drive mechanism into the structure of tissue sample holder (300). Such incorporated components may be desirable to reduce the form factor of probe (100) for use in operational environments where space may be occupied by other equipment used in a biopsy procedure.

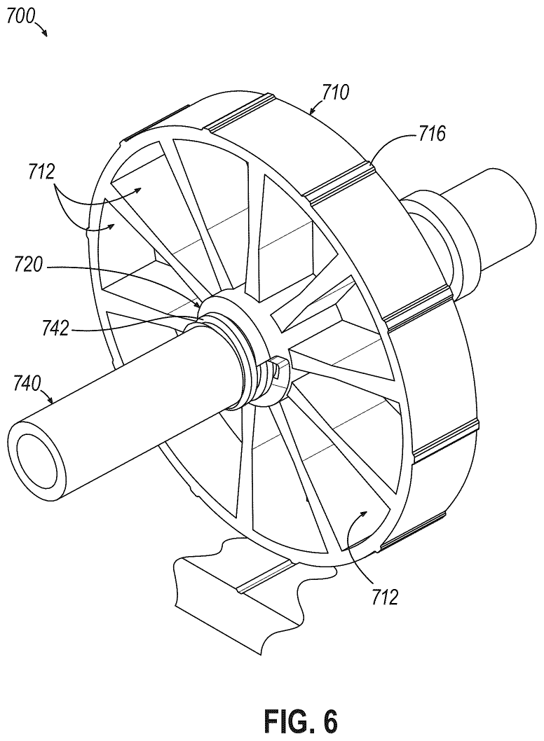

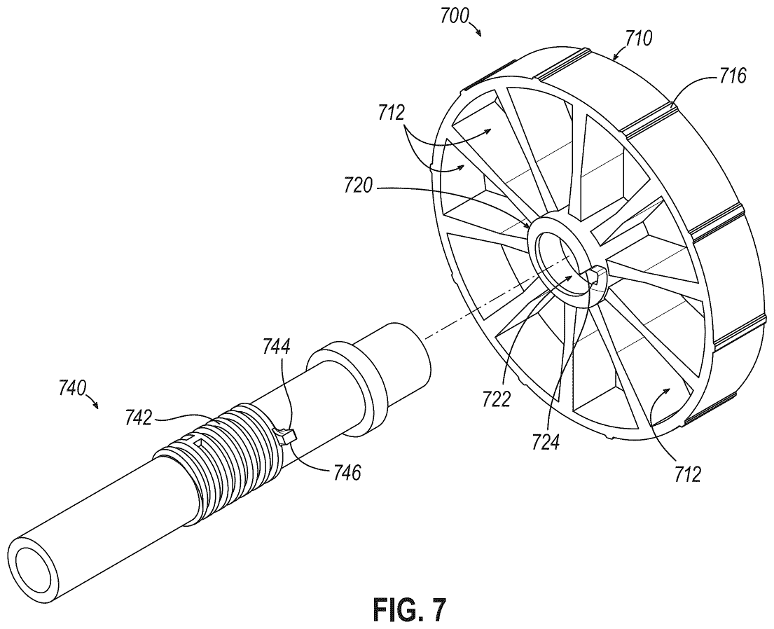

FIGS. 6 and 7 show an exemplary drive mechanism (700) that may be readily incorporated into probe (100) to drive movement of one or more components of tissue sample holder (300) such as manifold (310). Drive mechanism (700) of the present example is similar to drive mechanism (600) described above in that drive mechanism (700) is configured to rotate and translate one or more portions of tissue sample holder (300). However, unlike drive mechanism (600) described above, drive mechanism (700) of the present example combines features similar to rotation drive (610) and translation drive (620) into a single mechanism. Additionally, drive mechanism (700) of the present example is at least partially incorporated into tissue sample holder (300) itself rather than being a completely separate mechanism.

In the present example, drive mechanism (700) includes a drive element (720) and a drive screw (740). As best seen in FIG. 7, drive screw (740) is configured to be received within a portion of drive element (720). Drive screw (740) includes a threaded portion (742) and a hard stop (744) positioned on a proximal side of threaded portion (742). As will be described in greater detail below, threaded portion (742) and hard stop (744) are configured to engage drive element (720) during operation to generate a predetermined sequence of rotational and translational drive using rotation of drive screw (740).

Drive element (720) is generally incorporated into a portion of tissue sample holder (300). Specifically, drive element (720) is shown as being incorporated into a manifold (710) (also referred to as a rotatable member), which is substantially similar to manifold (310) described above. For instance, like with manifold (310) described above, manifold (710) of the present example defines a plurality of passages (712) that are configured to receive tissue receiving trays (330) or similar components. Thus, manifold (710) of the present example is generally configured for use similarly to manifold (310) described above to index passages (712) with cutter (150) so that tissue samples may be deposited within each tissue receiving tray (330).

Unlike manifold (310) described above, manifold (710) of the present example includes a plurality of detent features (716) disposed on an outer surface of manifold (710). As will be described in greater detail below, detent features (716) are generally configured to engage a portion of tissue sample holder (300) such as transparent cover (302) or a portion of probe (100) to releasably hold manifold (710) in one of a plurality of predetermined rotational positions. Thus, each detent feature (716) is generally aligned with a corresponding passage (712). Each detent feature (716) is defined by a semi-cylindrical protrusion oriented along the axis of rotation of manifold (710). In other examples, various alternative configurations for each detent feature (716) may be used such as semi-spherical shapes, triangular shapes, and/or etc. Each detent feature (716) may additionally be configured as either a protrusion or indentation.

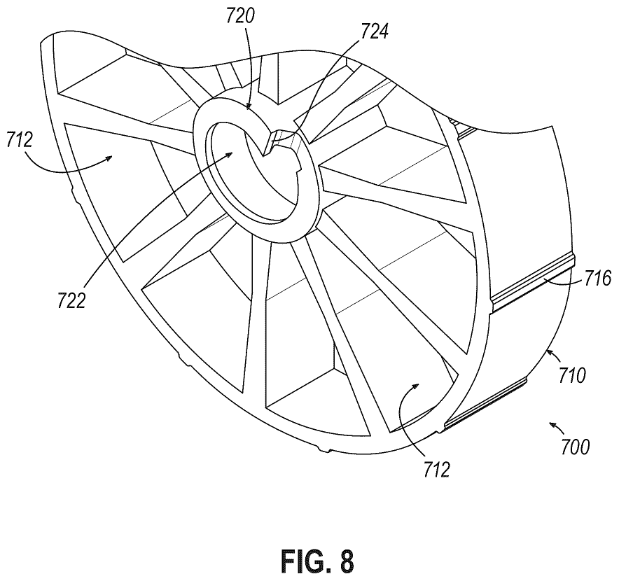

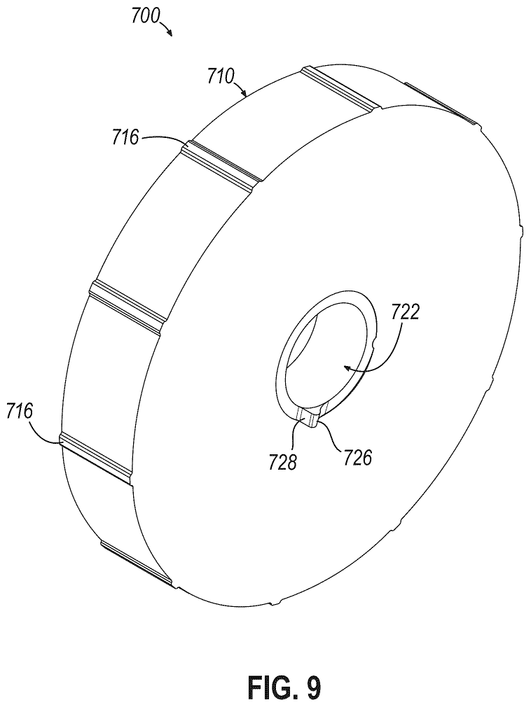

As best seen in FIGS. 8 and 9, drive element (720) includes a through bore (722) extending through the center of manifold (710), a threaded receiver (724) proximate through bore (722) and a hard stop (726) also proximate through bore (722). Through bore (722) is generally configured to receive drive screw (740) such that drive screw (740) extends entirely through manifold (710). Thus, through bore (722) is generally aligned with an axis of rotation of manifold (710) to facilitate rotation of manifold (710) via drive screw (740).

As best seen in FIG. 8, threaded receiver (724) is positioned on a distal side of manifold (710) proximate through bore (722). Threaded receiver (724) is generally configured to engage threaded portion (742) of drive screw (740) to convert rotation of drive screw (740) into translation of manifold (710), thereby moving manifold (710) relative to structures of probe (100) such as sealing member (170).

As best seen in FIG. 9, hard stop (726) is positioned on a proximal side of manifold (710) and extends proximally from a proximal edge thereof. Hard stop (726) includes a hard stop face (728) oriented perpendicularly relative to a rotation axis of manifold (710). Hard stop face (728) is configured to engage a corresponding hard stop face (746) of drive screw (740). As will be described in greater detail below, such engagement is used to transfer rotation of drive screw (740) to manifold (710) to thereby drive rotation of manifold (710) using drive screw (740). As will be appreciated, hard stops (726, 744) together with detent features (716) form a rotation stop that is configured to releasably hold manifold (710) in a predetermined rotational position relative to drive screw (740).

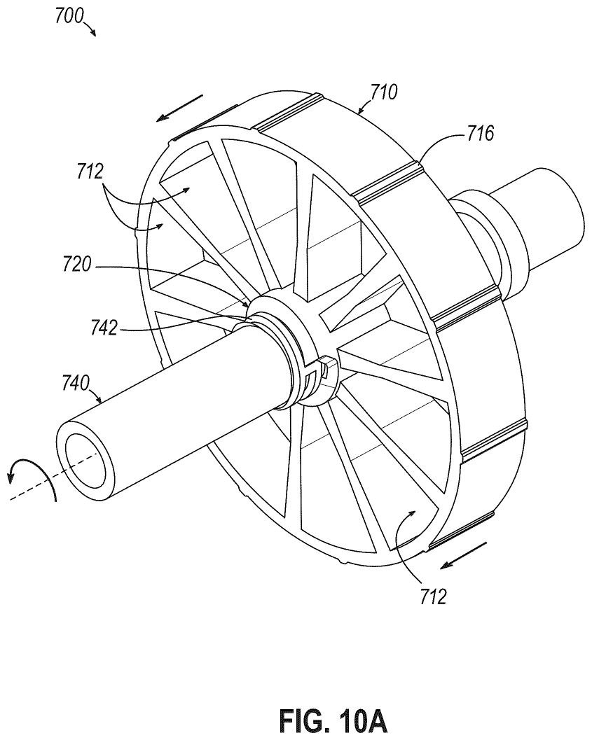

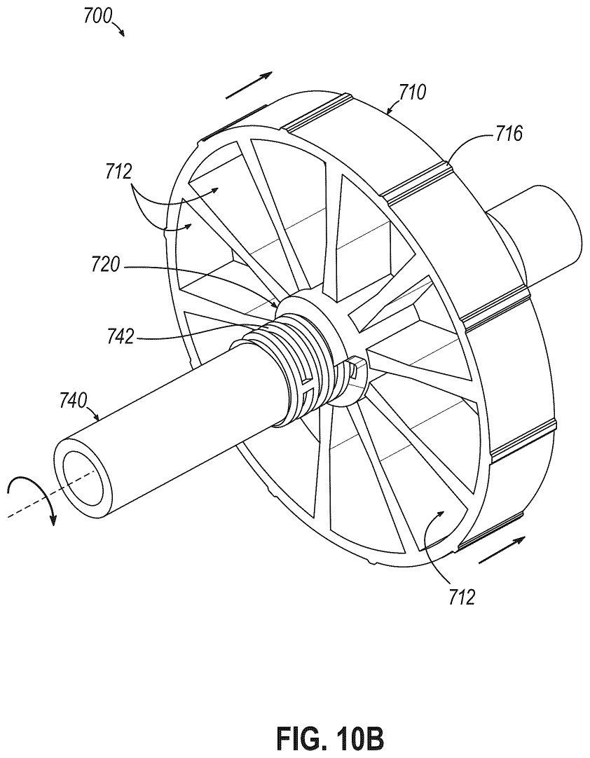

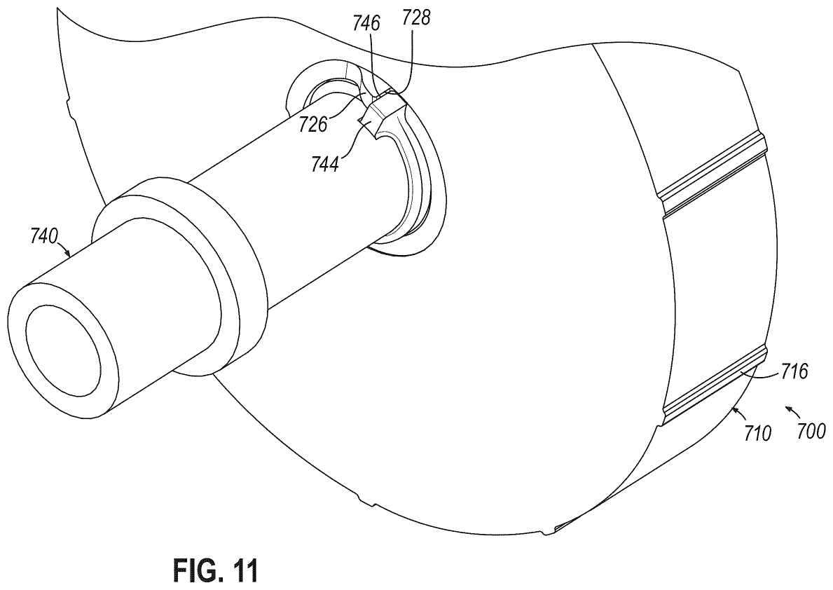

FIGS. 10A through 11 show an exemplary use of drive mechanism (700) to sequentially rotate and translate manifold (710). As best seen in FIG. 10A, drive screw (740) may be initially rotated in a counterclockwise direction. At this stage, engagement between detent features (716) and a portion of tissue sample holder (300) such as transparent cover (302) or a portion of probe (100) prevents corresponding counterclockwise rotation of manifold (710). Consequently, engagement between threaded receiver (724) of manifold (710) and threaded portion (742) of drive screw (740) drives distal translation of manifold (710). Although not shown, it should be understood that this distal translation of manifold (710) may drive manifold (710) into contact with a sealing feature such as seal (602), sealing member (170), or other alternative sealing features describe herein to seal manifold (710) relative to the exterior of tissue sample holder (300). In some uses, this sealing may correspond to collection of tissue samples into an indexed passage (712) of passages (712).

After sealing of manifold (710) via translation of manifold (710) by drive mechanism (700), it may be desirable to rotate manifold (710). For instance, after one or more tissue samples are received within an indexed passage (712), it may be desirable to index another passage (712) for receipt of additional tissue samples therein. To initiate rotation of manifold (710), drive screw (740) may be rotated in a reverse direction - a clockwise direction in the present example. As can be seen in FIG. 10B, clockwise rotation of drive screw (740) initially results in proximal translation of manifold (710). Specifically, engagement between detent features (716) and a portion of tissue sample holder (300) such as transparent cover (302) or a portion of probe (100) initially prevents rotation of manifold (710). Consequently, engagement between threaded receiver (724) of manifold (710) and threaded portion (742) of drive screw (740) drives proximal translation of manifold (710). Although not shown, this proximal translation of manifold (710) may drive manifold (710) away from, or out of contact with, a sealing feature such as seal (602), sealing member (170), or other alternative sealing features described herein. As a result, friction between manifold (710) and such sealing features may be reduced or entirely eliminated.

After at least some proximal translation of manifold (710), hard stop (744) of drive screw (740) engages hard stop (726) of manifold (710) as shown in FIG. 11. Upon engagement thereof, rotation of drive screw (740) is transferred to manifold (710) via engagement between hard stops (744, 726). Such transfer of rotation first overcomes engagement between detent features (716) and the portion of tissue sample holder (300) such as transparent cover (302) or the portion of probe (100). Upon disengagement of detent features (716), drive screw (740) drives rotation of manifold (710). Due to the proximal translation of manifold (710) prior to rotation of manifold (710), such rotation of manifold (710) may be with limited friction from the interface between manifold (710) and associated sealing features such as seal (602), sealing member (170), or other alternative sealing features described herein.

Rotation of manifold (710) may continue until detent features (716) reengage with the portion of tissue sample holder (300) such as transparent over (302) or the portion of probe (100). In some uses, this may correspond to indexing the next adjacent passage (712) with cutter (150). Alternatively, rotation of manifold (710) may continue through one or more engagement-disengagement cycles of detent features (716) until a particular predetermined passage (712) is indexed with cutter (150). Regardless, once a desired passage (712) is indexed with cutter (150), the same sequence of movement described above may be repeated to translate and seal manifold (710) and then rotate manifold (710) to index another passage (712). This sequence of movement may be repeated until all passages are occupied with one or more tissue samples or a desired number of tissue samples have been collected.

### C. Exemplary Alternative Tissue Sample Holder Drive Assembly With Manifold Integration

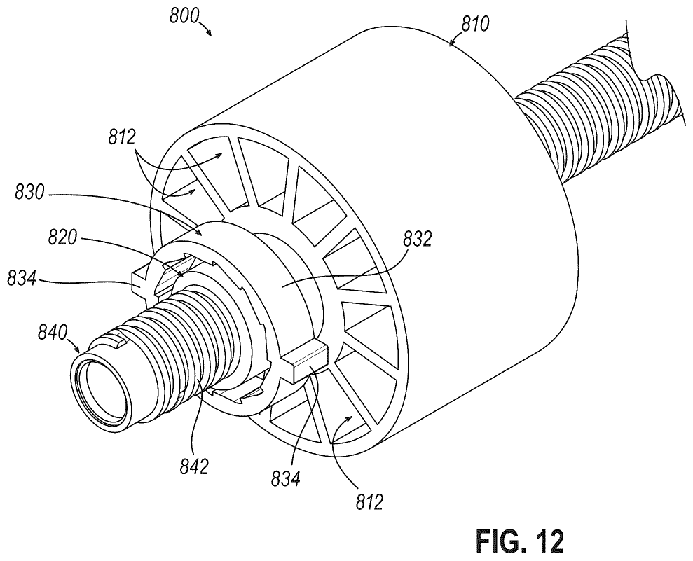

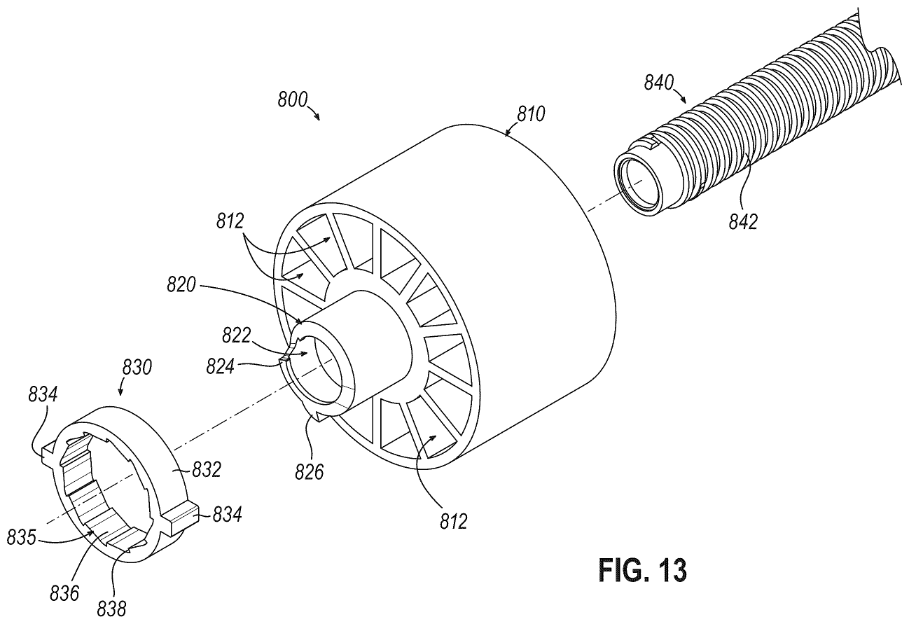

FIGS. 12 and 13 show an exemplary drive mechanism (800) that may be readily incorporated into probe (100) to drive movement of one or more components of tissue sample holder (300) such as manifold (310). Drive mechanism (800) of the present example is similar to drive mechanism (700) described above in that drive mechanism (800) is configured to rotate and translate one or more portions of tissue sample holder (300). Similarly, drive mechanism (800) of the present example combines features similar to rotation drive (610) and translation drive (620) into a single mechanism. Additionally, drive mechanism (800) of the present example is at least partially incorporated into tissue sample holder (300) itself rather than being a completely separate mechanism. However, unlike drive mechanism (700) described above, drive mechanism (800) includes a configuration that controls rotation and translation movements differently relative to drive mechanism (700), thereby omitting features such as detent features (716).

In the present example, drive mechanism (800) includes a drive element (820), a control element (830), and a drive screw (840). As best seen in FIG. 12, drive screw (840) is configured to be received within a portion of drive element (820). Meanwhile, control element (830) is configured to engage a portion of drive element (820). Drive screw (840) includes a threaded portion (842). As will be described in greater detail below, threaded portion (842) is configured to engage drive element (820) during operation to generate a predetermined sequence of rotational and translational drive using rotation of drive screw (840).

Drive element (820) is generally incorporated into a portion of tissue sample holder (300). Specifically, drive element (820) is shown as being incorporated into a manifold (810) (also referred to as a rotatable member), which is substantially similar to manifold (310) described above. For instance, like with manifold (310) described above, manifold (810) of the present example defines a plurality of passages (812) that are configured to receive tissue receiving trays (330) or similar components. Thus, manifold (810) of the present example is generally configured for use similarly to manifold (310) described above to index passages (812) with cutter (150) so that tissue samples may be deposited within each tissue receiving tray (330).

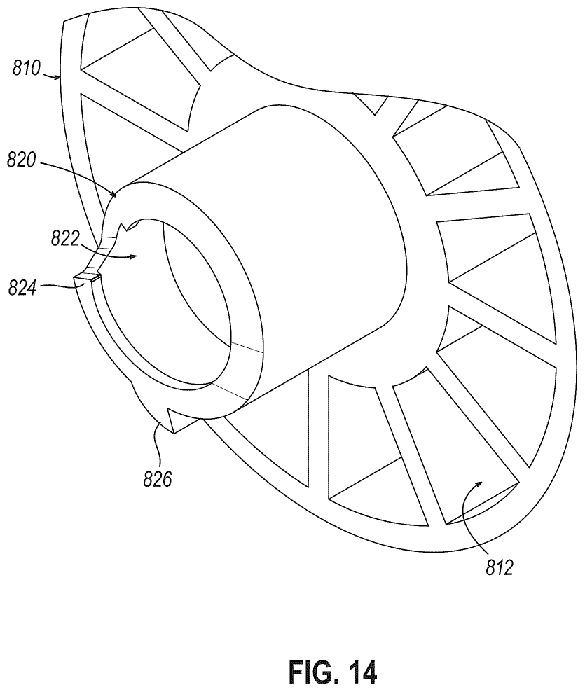

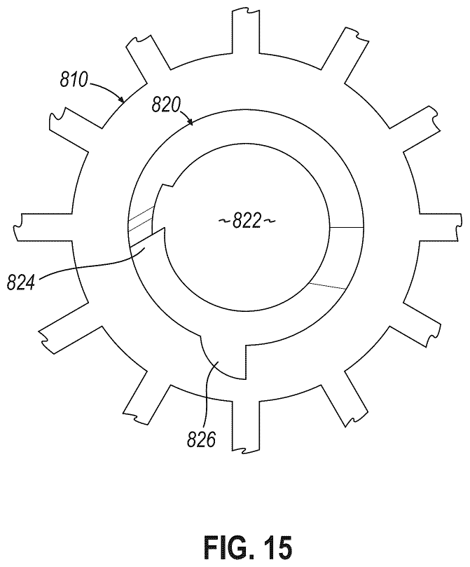

As best seen in FIGS. 14 and 15, drive element (820) includes a through bore (822) extending through the center of manifold (810), a threaded receiver (824) proximate through bore (822) and a stop member (826) also proximate through bore (822). Through bore (822) is generally configured to receive drive screw (840) such that drive screw (840) extends entirely through manifold (810). Thus, through bore (822) is generally aligned with an axis of rotation of manifold (810) to facilitate rotation of manifold (810) via drive screw (840).

As best seen in FIG. 14, threaded receiver (824) is positioned on a distal side of manifold (810) proximate through bore (822). Threaded receiver (824) is generally configured to engage threaded portion (842) of drive screw (840) to convert rotation of drive screw (840) into translation of manifold (810), thereby moving manifold (810) relative to structures of probe (100) such as sealing member (170).

Stop member (826) is also positioned on the distal side of manifold (810). As best seen in FIG. 15, stop member (826) also extends outwardly from a portion of threaded receiver (824). Stop member (826) includes an engagement face (828) oriented perpendicularly relative to a rotation axis of manifold (810). Engagement face (828) is configured to releasably engage one or more portions of control element (830). As will be described in greater detail below, such engagement is used to control engagement between threaded receiver (824) and drive screw (840) to control the transfer rotation of drive screw (840) to manifold (810).

Returning to FIG. 13, control element (830) includes a body (832) with one or more lock protrusions (834) extending outwardly from body (832). Although not shown, it should be understood that lock protrusions (834) are configured for receipt within a portion of probe (100) or tissue sample holder (300) to lock control element (830) in a fixed position relative to manifold (810).

Body (832) defines an annular structure with a stop surface (835) disposed on an interior surface of body (832). Body (832) is configured to receive drive element (820) within the annular structure to promote engagement between stop surface (835) and stop member (826) of drive element (820). Stop surface (835) defines as generally toothed structure similar to a cylindrical ratchet rack. Specifically, stop surface (835) includes a plurality of cam surfaces (836) and stop faces (838) interconnected together in an alternating pattern along the interior of the annular shape of body (832). As will be described in greater detail below, this configuration of stop surface (835) may permit rotation of manifold (810) in one direction after application of a sufficient force, while entirely preventing rotation of manifold (810) in another direction via engagement between stop surface (835) and stop member (826) of drive element (820). Thus, stop surface (835) together with stop member (826) form a rotation stop that is configured to releasably hold manifold (810) in a predetermined rotational position relative to drive screw (840).

Although a stop surface (835) is shown in the present example as having a particular configuration, it should be understood that other configurations may be used in other examples. For instance, in some examples, the ratchet structure of stop surface (835) may be replaced with detents, protrusions, indentations, or various combinations thereof. Of course, various alternative structures may be used for stop surface (835) as will be apparent to those of ordinary skill in the art in view of the teachings herein.

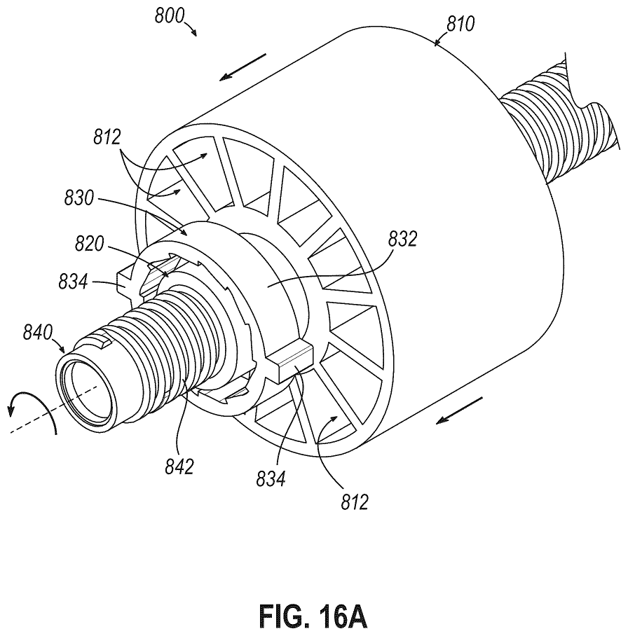

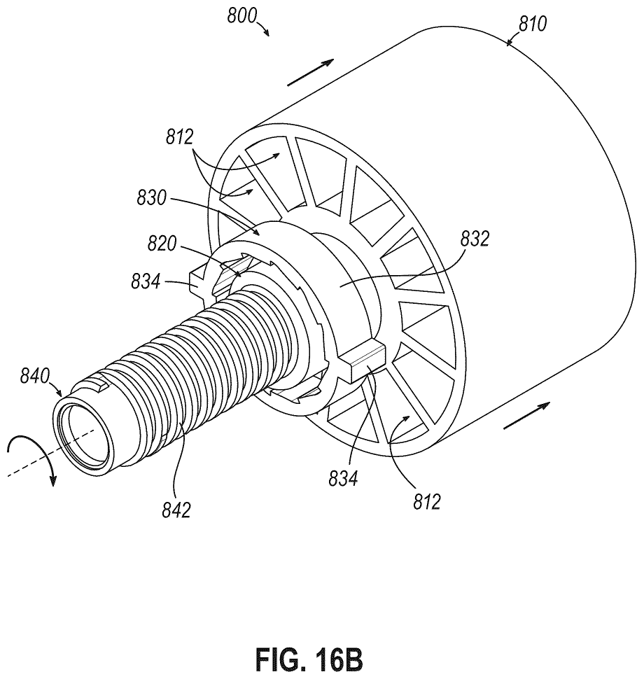

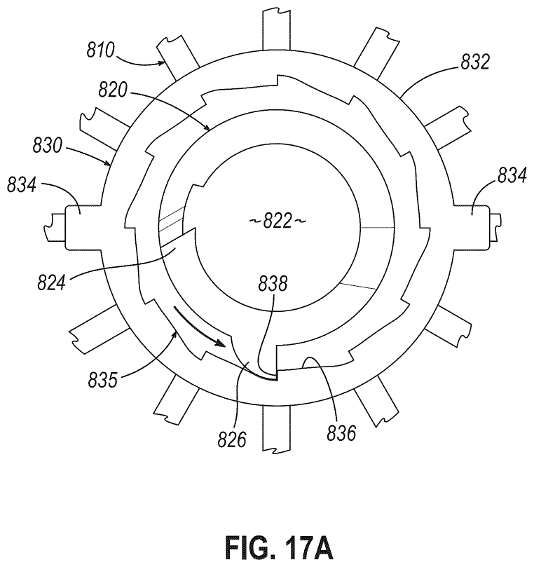

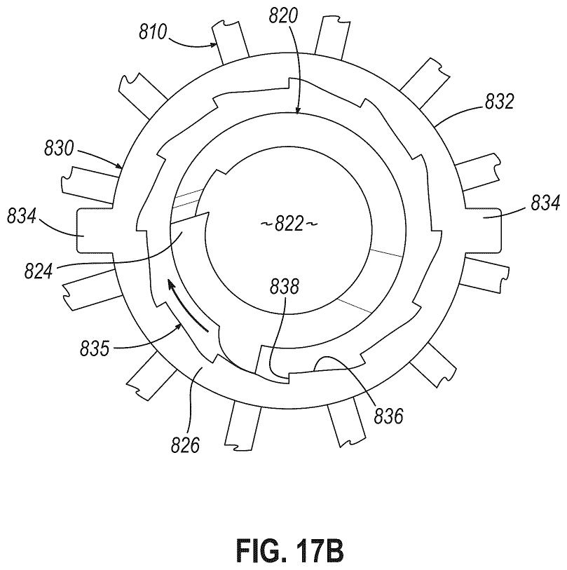

FIGS. 16A through 17B show an exemplary use of drive mechanism (800) to sequentially rotate and translate manifold (810). As best seen in FIG. 16A, drive screw (840) may be initially rotated in a counterclockwise direction. At this stage, engagement between stop member (826) and stop surface (835) of control element (830) prevent corresponding rotation of manifold (810). Specifically, as best seen in FIG. 17A, engagement face (828) of stop member (826) engages a given stop face (838) of stop surface (835). Both engagement face (828) and stop face (838) are generally oriented along parallel planes, thereby preventing relative rotational movement between control element (830) and manifold (810). Consequently, engagement between threaded receiver (824) of manifold (810) and threaded portion (842) of drive screw (840) drives distal translation of manifold (810). Although not shown, it should be understood that this distal translation of manifold (810) may drive manifold (810) into contact with a sealing feature such as seal (602), sealing member (170), or other alternative sealing features describe herein to seal manifold (810) relative to the exterior of tissue sample holder (300). In some uses, this sealing may correspond to collection of tissue samples into an indexed passage (812) of passages (812).

After sealing of manifold (810) via translation of manifold (810) by drive mechanism (800), it may be desirable to rotate manifold (810). For instance, after one or more tissue samples are received within an indexed passage (812), it may be desirable to index another passage (812) for receipt of additional tissue samples therein. To initiate rotation of manifold (810), drive screw (840) may be rotated in a reverse direction - a clockwise direction in the present example. As can be seen in FIG. 16B, clockwise rotation of drive screw (840) initially results in proximal translation of manifold (810). Specifically, engagement between stop member (826) and stop surface (835) of control element (830) is configured to temporarily prevent corresponding rotation of manifold (810). As best seen in FIG. 17B, stop member (826) is driven away from a given stop face (838) of stop surface (835) toward a given cam surface (836) of stop surface (835). The durometer of stop member (826), stop surface (835), or both may be configured to lock relative rotation between control element (830) and manifold (810) until a predetermined threshold is reached. In addition, or in the alternative, the profile of each cam surface (836) may be tuned to likewise lock relative rotation between control element (830) and manifold (810) until a predetermined threshold is reached. Consequently, engagement between threaded receiver (824) of manifold (810) and threaded portion (842) of drive screw (840) drives proximal translation of manifold (810). Although not shown, this proximal translation of manifold (810) may drive manifold (810) away from, or out of contact with, a sealing feature such as seal (602), sealing member (170), or other alternative sealing features described herein. As a result, friction between manifold (810) and such sealing features may be reduced or entirely eliminated. In other uses, translation of manifold (810) may be limited and the translation action at this stage may instead be characterized as a release of a force between manifold (810) and associated sealing features.

After at least some proximal translation of manifold (810) (or reduction in force between manifold (810) and sealing features associated with manifold (810), drive screw (840) is rotated further in the clockwise direction to correspondingly rotate manifold (810). Specifically, after a predetermined force threshold is reached between drive element (820) and a given cam surface (836), drive element (820) is driven along the given cam surface (836) as best seen in FIG. 17B. Such movement may be accomplished by friction between threaded portion (842) of drive screw (840) and threaded receiver (824) of drive element (820), thereby transferring rotational motion from drive screw (840) to manifold (810).

Rotation of manifold (810) may continue until stop member (826) moves from a given cam surface (836) to the next stop face (838) of control element (830). In some uses, this may correspond to indexing the next adjacent passage (812) with cutter (150). Alternatively, in some uses, rotation of manifold (810) may continue through one or more engagement-disengagement cycles of cam surfaces (836) and stop faces (838) until a particular predetermined passage (812) is indexed with cutter (150). Regardless, once a desired passage (812) is indexed with cutter (150), the same sequence of movement described above may be repeated to translate and seal manifold (810) and then rotate manifold (810) to index another passage (812). This sequence of movement may be repeated until all passages are occupied with one or more tissue samples or a desired number of tissue samples have been collected.

### IV. Exemplary Alternative Tissue Sample Holder With Seal Lock Mechanism

In some drive mechanisms similar to drive mechanisms (600, 700, 800) described above, it may be desirable to drive different movements using separate mechanisms or components. For instance, in use with a tissue sample holder similar to tissue sample holder (300) described above, it may be desirable to drive translation of structures similar to manifold (310) separately from driving rotation of such structures. Such separation of movement drive may be desirable to provide enhanced control over the specific motions involved. In addition, such separation of movement drive may be desirable to simplify the drive mechanisms involved. Furthermore, such separation of movement drive may be desirable to permit alternative configurations of structures similar to manifold (310), which may enhance use of the tissue sample holder. Although certain features are described below are described in the context of being used to drive translation separately of rotation, it should be understood that in other examples such features may be used in combination with various rotation mechanisms where rotational and translational movements may be linked. In some examples one or more features described below may be combined with features of one or more alternative drive mechanisms such as drive mechanisms (600, 700, 800) described above.

### A. Exemplary Alternative Tissue Sample Holder With Cam-Based Seal Lock Mechanism

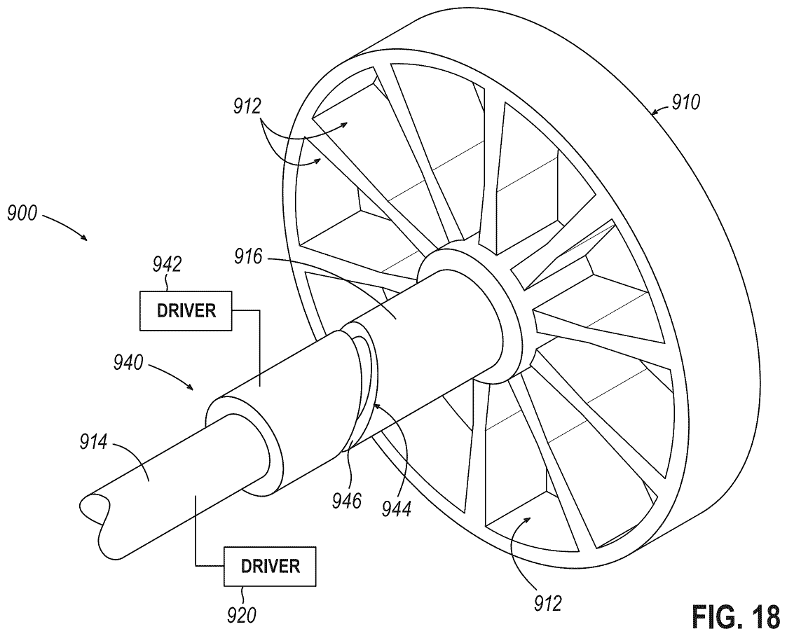

FIG. 18 shows an exemplary drive mechanism (900) (also referred to as a tissue sample holder drive) that may be readily incorporated into probe (100) described above to facilitate movement of tissue sample holder (300). In the present example, drive mechanism (900) is generally configured to drive a portion of tissue sample holder (300) with both rotational motion and at least some translational motion relative to a sealing feature such as sealing member (170), seal (602), or other sealing structures described herein. As will be described in greater detail below, such rotational and translational motions may facilitate indexing of tissue sample holder (300) relative to cutter (150), while also reducing friction between sealing features associated with tissue sample holder (300) and one or more portions of tissue sample holder (300).

Drive mechanism (900) includes a rotation drive (920) and a translation drive (940). Both rotation drive (920) and translation drive (940) are in communication with a manifold (910) (also referred to as a rotatable member), which is substantially similar to manifold (310) described above. For instance, like with manifold (310) described above, manifold (910) of the present example defines a plurality of passages (912) that are configured to receive tissue receiving trays (330) or similar components. Thus, manifold (910) of the present example is generally configured for use similarly to manifold (310) described above to index passages (912) with cutter (150) so that tissue samples may be deposited within each tissue receiving tray (330).

Although rotation drive (920) and translation drive (940) are shown separately in the present example, it should be understood that in other examples, rotation drive (920) and translation drive (940) are incorporated into a single drive with a single communication pathway to tissue sample holder (300). In addition, or in the alternative, in some examples, rotation drive (920) and translation drive (940) may be separate, but in communication with each other to facilitate coordination of movements between rotation drive (920) and translation drive (940). Additionally, as will be described in greater detail below, in some examples one or more components of rotation drive (920) and/or translation drive (940) are readily incorporated into one or more components of tissue sample holder (300) such as manifold (310) or manifold (910).

Rotation drive (920) is generally configured to drive rotation of manifold (910). Thus, rotation drive (920) is in communication with manifold (910) via an inner rotation shaft (914) to communicate rotatory power to manifold (910). Although not shown, it should be understood that rotation drive (920) may include a variety of components configured to drive rotation of inner rotation shaft (914). By way of example only, suitable components for use with rotation drive (920) may include one or more shafts, gears, transmissions, motors, and/or etc.

Translation drive (940) is generally configured to drive translation of manifold (910) proximally and distally. In the present example, such translation is entirely independent of rotation of manifold (910) driven by rotation drive (920). In other examples, such translation is sequential and is coordinated with rotation driven by rotation drive (920). As will be described in greater detail below, the particular magnitude of translation driven by translation drive (940) is an amount sufficient to reduce friction at an interface between manifold (910) one or more sealing features associated with manifold (910) such as sealing member (170), seal (602), or other sealing features described herein. In some examples, the magnitude of translation driven by translation drive (940) corresponds to 1 to 4 mm. In other examples, other suitable magnitudes of translation may be used as will be apparent to those of ordinary skill in the art in view of the teachings herein.

Translation drive (940) is in communication with manifold (910) via an outer translation shaft (916) extending distally from a distal face of manifold (910). Outer translation shaft (916) is generally disposed on an exterior of inner rotation shaft (914) such that outer translation shaft (916) and inner rotation shaft (914) are coaxial. Thus, in the present example, outer translation shaft (916) is hollow and configured to receive at least a portion of inner rotation shaft (914). Although outer translation shaft (916) is configured as an outer shaft in the present example, it should be understood that in other examples, the configuration is reversed with outer translation shaft (916) being received within inner rotation shaft (914). Thus, outer translation shaft (916) and inner rotation shaft (914) may be referred to as simply translation shaft and rotation shaft in some examples. Additionally, although outer translation shaft (916) and inner rotation shaft (914) are coaxial in the present example, in other examples outer translation shaft (916) and inner rotation shaft (914) are disposed along separate axes.

Translation drive (940) includes a driver (942) and a barrel cam (944). Generally, driver (942) and barrel cam (944) are configured to operate cooperatively to convert rotation of barrel cam (944) into translation of outer translation shaft (916), which drives translation of manifold (910). Driver (942) is generally configured to rotate barrel cam (944), outer translation shaft (916), or both. Thus, driver (942) includes a variety of components configured to facilitate rotary motion. By way of example only, suitable components for driver (942) include one or more shafts, gears, transmissions, motors, and/or etc.

Barrel cam (944) in the present example is integrated into a portion of outer translation shaft (916). In other examples, barrel cam (944) is a separate component fixedly secured or otherwise attached to a portion of outer translation shaft (916). It some examples, barrel cam (944) is configured to rotate relative to outer translation shaft (916), while remaining in a fixed axial position along the length of outer translation shaft (916). For instance, in some examples, outer translation shaft (916) is fixed relative to manifold (910) so that rotation of outer translation shaft (916) results in corresponding rotation of manifold (910). In such examples, it may be desirable to decouple rotation of barrel cam (944) from rotation of outer translation shaft (916) to prevent transferring rotation of barrel cam (944) to manifold (910) via outer translation shaft (916). In other examples, outer translation shaft (916) includes one or more coupling features configured to permit rotation of outer translation shaft (916) relative to manifold (910). In such versions, barrel cam (944) may be integral with, or fixedly secured to, outer translation shaft (916).

Regardless of the particular relationship between barrel cam (944) and outer translation shaft (916), barrel cam (944) defines a cam path (946) extending into and around the outer surface of barrel cam (944). Cam path (946) is generally configured to engage with a cam follower (950) or other feature to guide barrel cam (944) through a predetermined translation path. In the present example, cam path (946) extends entirely around the circumference of barrel cam (944). In other examples, cam path (946) extends around only a portion of the circumference of barrel cam (944).

Cam path (946) generally defines a sweeping path from a distal inflection to a proximal inflection. Thus, cam path (946) is configured such that a full rotation of barrel cam (944) results in a full translational movement of manifold (910) either distally or proximally depending on the direction of rotation of barrel cam (944). Accordingly, the particular distance between the distal inflection and the proximal inflection defines the total translation distance for manifold (910). It should be understood that the particular sweep or curvature of cam path (946) may be varied and may have relationship with the distance between the distal inflection and the proximal inflection. For instance, in some examples, cam path (946) includes a curve of relatively low slope, which corresponds to the distance between distal inflection and proximal inflection is relatively small. Similarly, in other examples, cam path (946) includes a curve of relatively high slope, which corresponds to the distance between distal inflection and proximal inflection is relatively large. In addition, or in the alternative, in some examples, the slope defined by cam path (946) is relatively consistent. In other examples, the slope defined by cam path (946) varies over its length. Such varying slopes may be desirable in some circumstances to vary the mechanical advantage of barrel cam (944) over the translation path of manifold (910). Of course, various other alternative configurations for cam path (946) may be used as will be apparent to those of ordinary skill in the art in view of the teachings herein.

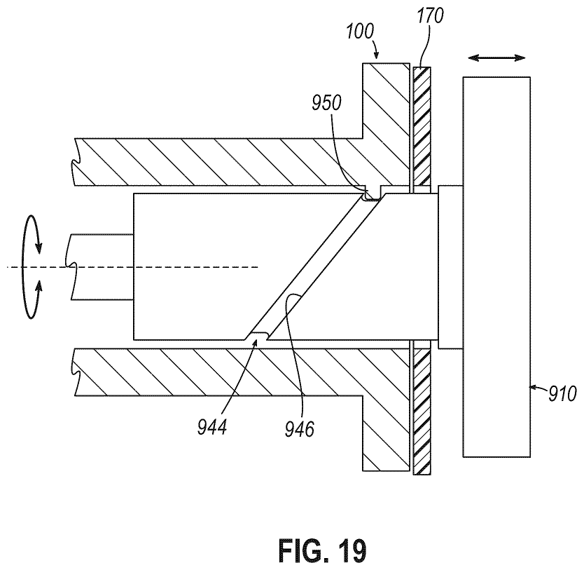

FIG. 19 shows an exemplary use of drive mechanism (900) to translate manifold (910). As can be seen, barrel cam (944) may be rotated either in the clockwise or counterclockwise direction by barrel cam (944). During rotation, cam follower (950), which is fixedly secured to a portion of probe (100) or one or more other structures, engages cam path (946) of barrel cam (944). As barrel cam (944) rotates, this engagement causes barrel cam (944) to be driven by cam follower (950) along the path defines by cam path (946). Consequently, barrel cam (944) translates proximally or distally depending on the direction of rotation of barrel cam (944). Manifold (910) then translates via outer translation shaft (916).

In use with probe (100), translation of manifold (910) via barrel cam (944) may correspond to circumstances where it may be desirable to move manifold (910) between a sealed and unsealed stage. For instance, during collection of tissue samples, it may be desirable to seal manifold (910) relative to probe (100). Thus, drive mechanism (900) may be used to translate manifold (910) distally into one or more sealing feature such as sealing member (170), seal (602), or other sealing features described herein. After collection of one or more tissue samples within a passage (912) of manifold (910), it may next be desirable to index another passage (912) with cutter (150). Thus, drive mechanism (900) may be used to translate manifold (910) proximally away from such one or more sealing features to facilitate ease of rotation via rotation drive (920). Rotation drive (920) may then be used to rotate manifold (910) to index another passage (912) with cutter (150). The same process may then be repeated until a desired number of tissue samples are collected.

### B. Exemplary Alternative Tissue Sample Holder With Rotational Seal Lock

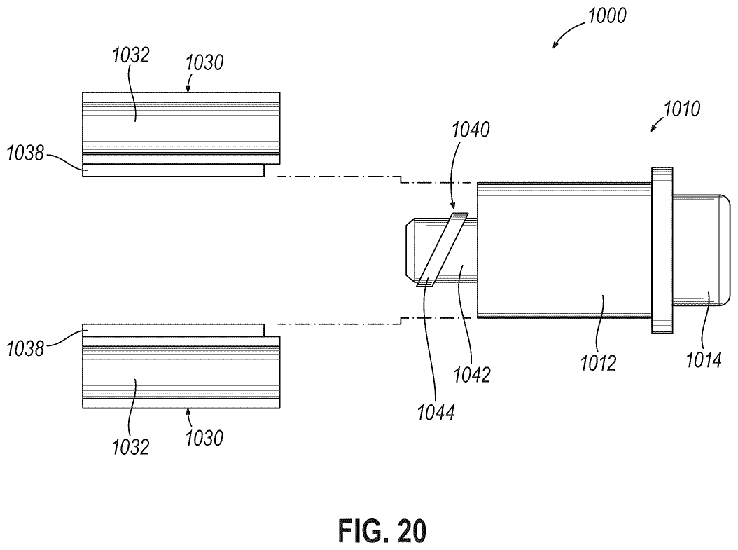

FIG. 20 shows an exemplary alternative tissue sample holder (1000) with an integral translation drive (1040). Tissue sample holder (1000) of the present example is configured for use with probe (100) in lieu of tissue sample holder (300) described above. Tissue sample holder (1000) of the present example is similar to tissue sample holder (300) described above in that tissue sample holder (1000) is rotatable relative to probe (100) to index a plurality of tissue sample chambers (1036) of one or more tissue receiving trays (1030) relative to cutter (150). However, unlike tissue sample holder (300) described above, tissue sample holder (1000) of the present example includes a tray retainer (1010) (also referred to as a rotatable member or lock collar) that is configured to receive one or more tissue receiving trays (1030) rather than a manifold similar to manifold (310) described above. As will be described in greater detail below, tray retainer (1010) is configured to both receive one or more tissue receiving trays (1030) and translate distally and proximally relative to probe (100) to permit sealing of one or more tissue receiving trays (1030). Although tray retainer (1010) is characterized herein as a “tray retainer,” it should be understood that in some contexts, tray retainer (1010) may be characterized as a modified version of manifold (310) described above.

Tray retainer (1010) includes a cylindrical body (1012), a rotation tab (1014), and one or more tray receiving features (1016). As will be described in greater detail below, tray retainer (1010) is generally configured to releasably receive a portion of each tissue receiving tray (1030) within tray receiving features (1016) to releasably secure each tissue receiving tray (1030) to tray retainer (1010). Tray retainer (1010) is further generally configured to rotate relative to probe (100) via rotation tab (1014) to drive translation of tray retainer (1010) and one or more tissue receiving trays (1030) to selectively seal one or more tissue receiving trays (1030) relative to probe (100).

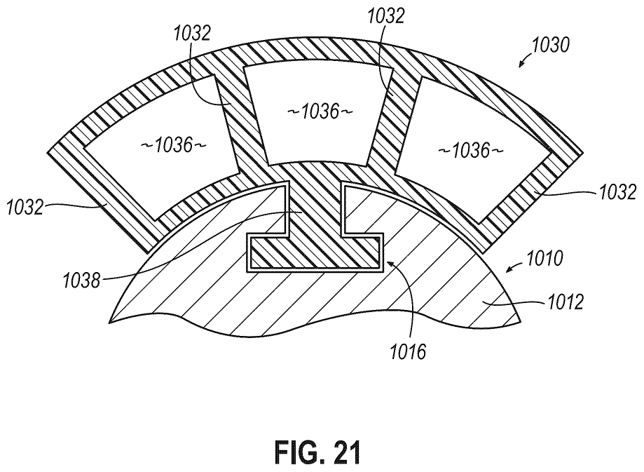

As best seen in FIG. 21, each tray receiving feature (1016) is defined by tray retainer (1010) as a keyed channel extending axially though cylindrical body (1012). Although only a single tray receiving feature (1016) is shown in FIG. 21, it should be understood that tray retainer (1010) includes a plurality of tray receiving features (1016). The particular number of tray receiving features (1016) used generally corresponds to the particular number of tissue receiving trays (1030) used. Thus, in examples with three tissue receiving trays (1030), tray retainer (1010) likewise defines three tray receiving features (1016). Alternatively, in other examples, the ratio of tray receiving features (1016) to tissue receiving trays (1030) can be varied. For instance, in some examples, two tray receiving features (1016) are used to receive a single tissue receiving tray (1030). Thus, in the example above with three tissue receiving trays (1030), six tissue receiving features (1016) are used instead of three.

In the present example, the particular keyed channel defining each tray receiving feature (1016) is generally rectangular. Specifically, a relatively narrow rectangle is positioned above a relatively wide rectangle. In other examples, various alternative keyed channels are used. For instance, in some examples, various triangular, rounded, semi-circular, oval-shaped, or other keyed forms are used. Regardless of the particular form, it should be understood that in some examples, the dimensions of each tray receiving feature (1016) may be controlled relative to the dimensions of corresponding features of each tissue receiving tray (1030) to optionally provide an interference fit.

Tissue receiving trays (1030) of the present example are generally configured similarly to tissue receiving trays (330) described above. For instance, as with tissue receiving trays (330), tissue receiving trays (1030) of the present example include sidewalls (1032) and other similar structures (e.g., floor, end walls) that define a plurality of tissue sample chambers (1030). Similarly, tissue receiving trays (1030) define an opening (not shown) corresponding to each tissue sample chamber (1030) that are configured to receive one or more tissue samples from cutter (150).

Unlike tissue receiving trays (330) described above, tissue receiving trays (1030) of the present example each include a fastener (1038) projecting from a bottom surface of each tissue receiving tray (1030). Each fastener (1038) is configured to mate with a corresponding tray receiving feature (1016) of tray retainer (1010) to releasably secure each tissue receiving tray (1030) to tray retainer (1010). Thus, each fastener (1038) generally defines a shape corresponding to the shape of tray receiving features (1016). In the present example, the shape of each fastener (1038) is generally a keyed rectangular protrusion with a relatively narrow rectangular near the bottom surface of each tissue receiving tray (1030) and a relatively wide rectangular structure further from the bottom surface of each tissue receiving tray (1030). As described above, the particular shape of each tray receiving feature (1016) may be varied in some examples. Thus, the particular shape of each fastener (1038) may likewise be varied as described above to correspond to the particular shape of each tray receiving feature (1016).

Although not show, it should be understood that in some examples, tissue receiving trays (1030) additionally include one or more lumens extending through a portion thereof. Such lumens may be desirable to facilitate the flow of vacuum through each tissue receiving tray (1030) and into one or more tissue sample chambers (1036). By way of example only, such lumens may be positioned proximate fastener (1038) to facilitate the flow of vacuum through the bottom of each tissue receiving tray (1030). Alternatively, in other examples, one or more of such lumens are incorporated into tray retainer (1010) to supply vacuum via tray retainer (1010) directly or indirectly instead of directly through tissue receiving trays (1030).

Tissue sample holder (1000) further includes translation drive (1040). Translation drive (1040) is generally configured to engage a portion of probe (100) to drive translation of tray retainer (1010) distally and proximally relative to probe (100). Translation drive (1040) includes a translation shaft (1042) extending distally from a distal end of tray retainer (1010) and a threaded portion (1044) disposed on translation shaft (1042). Translation shaft (1042) and threaded portion (1044) are configured to operate cooperatively to engage a portion of probe (100) to drive translation of tray retainer (1010) distally and proximally via rotation of tray retainer (1010) via rotation tab (1014).

Translation drive (1040) is configured to engage probe (100) in a variety of ways. For instance, in the present example, translation drive (1040) is configured to engage a threaded portion of probe (100) corresponding to threaded portion (1044) of translation drive (1040). In some examples, such a threaded portion of probe (100) is incorporated into various tissue sample holder rotation structures such as gasping feature (184) described above. Thus, gasping feature (184) is configured to couple tissue sample holder (1000) to probe (100) via threaded portion (1044) of translation drive (1040). Additionally, gasping feature (184) includes other features to facilitate rotation of tissue sample holder (1000) once tissue sample holder (1000) is coupled to probe (100). In other examples, translation drive (1040) is configured to couple to other features of probe (100) unassociated with rotation of tissue sample holder (1000).

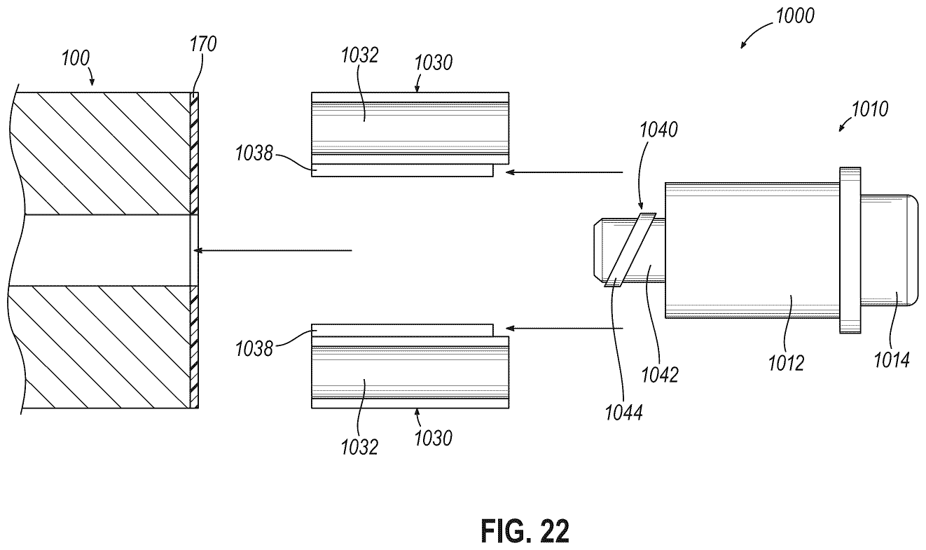

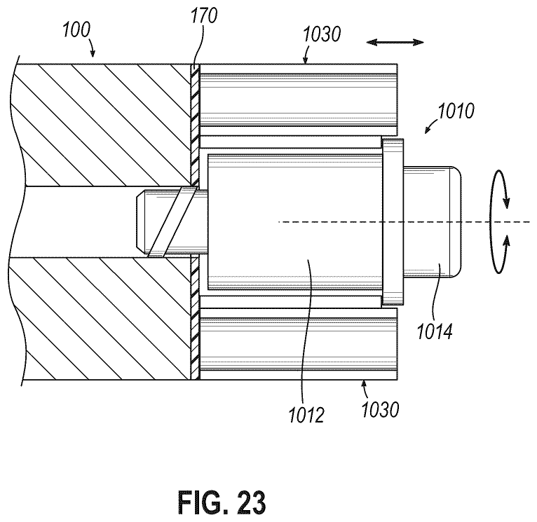

FIGS. 22 and 23 show an exemplary use of tissue sample holder (1000) in combination with probe (100) to translate at least a portion of tissue sample holder (1000) relative to probe (100) for sealing of tissue receiving trays (1030) relative to probe (100). As best seen in FIG. 22, tray retainer (1010), tissue receiving trays (1030), and probe (100) are initially separated from each other. At this stage, tray retainer (1010) and tissue receiving trays (1030) are manipulated relative to each other to insert tissue receiving trays (1030) into tray retainer (1010). Specifically, fastener (1038) of each tissue receiving tray (1030) is inserted into a corresponding tray receiving feature (1016) and each tissue receiving tray (1030) is slide axially onto tray retainer (1010).

After tissue receiving trays (1030) are secured to tray retainer (1010), the combination of tray retainer (1010) and tissue receiving trays (1030) may be secured to probe (100). Specifically, tray retainer (1010) is manipulated to engage threaded portion (1044) of translation drive (1040) with a corresponding threaded portion of probe (100). Tray retainer (1010) is then rotated via rotation tab (1014) relative to probe (100) to further engage threaded portion (1044) with the corresponding threaded portion of probe (100).

Once tissue sample holder (1000) is secured to probe (100), it may be desirable to seal tissue receiving trays (1030) relative to probe (100). Such sealing is achieved by further rotating tray retainer (1010) via rotation tab (1014) to translate tray retainer (1010) distally, thereby driving the distal end of tissue receiving trays (1030) into a sealing feature of probe (100) such as sealing member (170), seal (602), or other sealing features described herein.

Alternatively, prior to sealing, tissue sample holder (1000) is rotated relative to probe (100) via rotation feature of probe (100) such as grasping feature (184). Rotation of tissue sample holder (1000) at this stage may be desirable to initially index a particular tissue sample chamber (1036) with cutter (150) of probe (100). After such indexing, tissue receiving trays (1030) may be sealed by rotating tray retainer (1010) further via rotation tab (1014) as described above.

At one or more stages during a biopsy procedure, it may be desirable to rotate tissue sample holder (1000) after tissue receiving trays (1030) are sealed as described above. Prior to such rotation, it may be desirable to release the seal between tissue receiving trays (1030) and probe (100). To release such a seal, tray retainer (1010) is rotated in an opposite direction via rotation tab (1014). Such opposite rotation drives tray retainer (1010) in a proximal direction via threaded portion (1044). The amount of rotation of tray retainer (1010) at this stage is a degree of rotation sufficient to release the seal, while being insufficient to fully decouple tissue sample holder (1000) from probe (100). Tissue sample holder (1000) may then be rotated via one or more features of probe (100).

### C. Exemplary Alternative Tissue Sample Holder With Lock Collar for Seal Lock

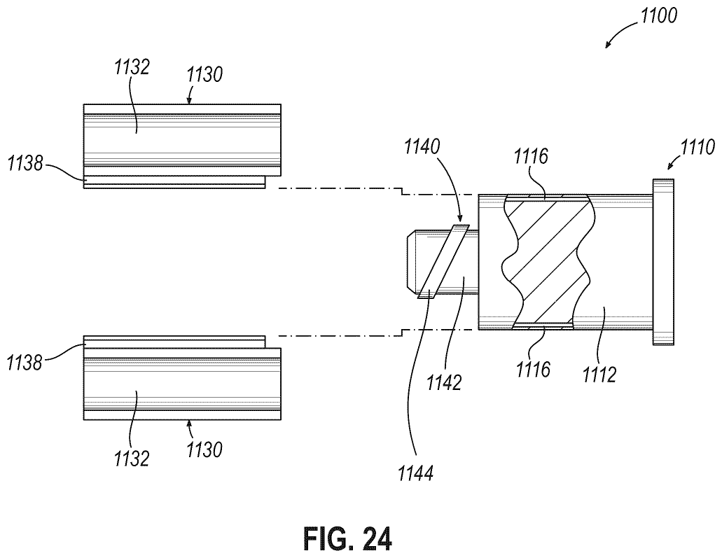

FIG. 24 shows another exemplary alternative tissue sample holder (1100) with an integral translation drive (1140). Tissue sample holder (1100) of the present example is configured for use with probe (100) in lieu of tissue sample holders (300, 1000) described above. Tissue sample holder (1100) of the present example is similar to tissue sample holder (300) described above in that tissue sample holder (1100) is rotatable relative to probe (100) to index a plurality of tissue sample chambers (1136) of one or more tissue receiving trays (1130) relative to cutter (150). However, unlike tissue sample holder (300) described above, tissue sample holder (1100) of the present example includes a tray retainer (1110) (also referred to as a rotatable member or lock collar) that is configured to receive one or more tissue receiving trays (1130) rather than a manifold similar to manifold (310) described above. As will be described in greater detail below, tray retainer (1110) is configured to both receive one or more tissue receiving trays (1130) and translate distally and proximally relative to probe (100) to permit sealing of one or more tissue receiving trays (1130). Although tray retainer (1110) is characterized herein as a “tray retainer,” it should be understood that in some contexts, tray retainer (1110) may be characterized as a modified version of manifold (310) described above.

Tray retainer (1110) of the present example is substantially similar to tray retainer (1010) described above with respect to tissue sample holder (1000). For instance, like with tray retainer (1010), tray retainer (1110) of the present example includes a cylindrical body (1112), and one or more tray receiving features (1116). Similarly to tray retainer (1010) described above, tray retainer (1110) of the present example is generally configured to releasably receive a portion of each tissue receiving tray (1130) within tray receiving features (1116) to releasably secure each tissue receiving tray (1130) to tray retainer (1110). However, unlike tray retainer (1010) described above, translation of tray retailer (1110) is driven separately from tissue sample holder (1100). Thus, features similar to rotation tab (1014) are omitted in the present example.

Each tray receiving feature (1116) of tray retainer (1110) is configured substantially similarly to tray receiving features (1016) described above. As similarly described above, each tray receiving feature (1116) is configured as a keyed channel extending axially though cylindrical body (1112). As also similarly described above, each tray receiving feature (1116) can be configured in a variety of shapes and configurations.

Tissue receiving trays (1130) of the present example are generally configured similarly to tissue receiving trays (1030) described above. For instance, as with tissue receiving trays (1030), tissue receiving trays (1130) of the present example include sidewalls (1132) and other similar structures (e.g., floor, end walls) that define a plurality of tissue sample chambers (not shown). Similarly, tissue receiving trays (1130) define an opening (not shown) corresponding to each tissue sample chamber (1130) that are configured to receive one or more tissue samples from cutter (150). Tissue receiving trays (1130) of the present example additionally each include a fastener (1138) projecting from a bottom surface of each tissue receiving tray (1130). Each fastener (1138) is configured substantially similarly to fasteners (1038) described above.

Tissue sample holder (1100) further includes translation drive (1140). Translation drive (1140) is generally configured to engage a portion of probe (100) to drive translation of tray retainer (1110) distally and proximally relative to probe (100). Translation drive (1140) includes a translation shaft (1142) extending distally from a distal end of tray retainer (1110) and a threaded portion (1144) disposed on translation shaft (1142). Translation shaft (1142) and threaded portion (1144) are configured to operate cooperatively to engage a portion of probe (100) to drive translation of tray retainer (1110) distally and proximally via rotation of tray retainer (1110).

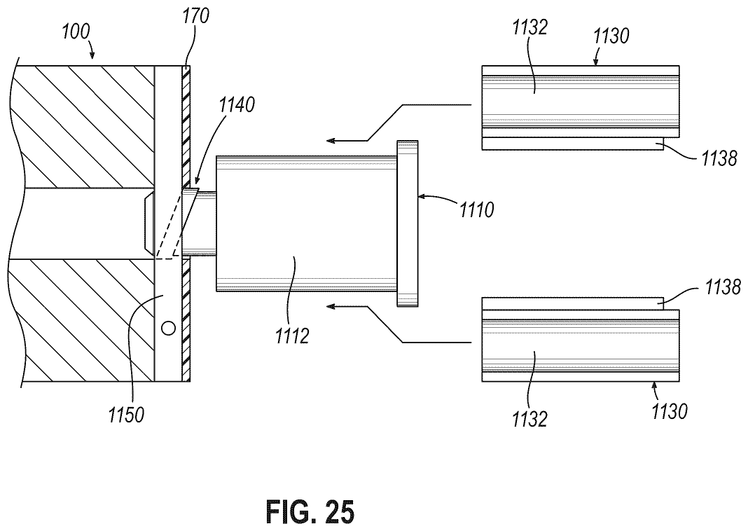

Unlike translation drive (1040) described above, translation drive (1140) of the present example further includes a translator (1150) as shown in FIG. 25. Translator (1150) is generally configured to engage translation shaft (1142) and threaded portion (1144) to drive distal and proximal translation of tray retainer (1110). In the present example, translator (1150) is incorporated into a proximal portion of probe (100) and is in the form of a collar. Although not shown, it should be understood that the interior of translator (1150) includes a threaded portion corresponding to threaded portion (1144) of translation shaft (1142). Thus, translator (1150) is configured to rotate or otherwise move relative to probe (100) to engage threaded portion (1144), thereby driving translation of tray retainer (1110). Although translator (1150) is configured as a collar in the present example, it should be understood that in other examples, translator (1150) may include a variety of forms such as levers, lock arms, wheels, and/or etc.

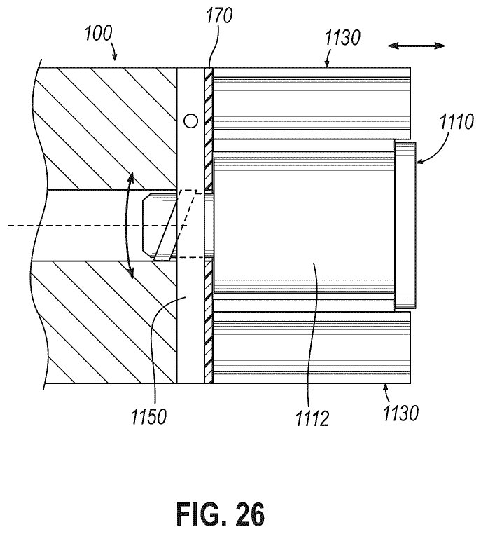

FIGS. 25 and 26 show an exemplary use of tissue sample holder (1100) in combination with probe (100) to translate at least a portion of tissue sample holder (1100) relative to probe (100) for sealing of tissue receiving trays (1130) relative to probe (100). As best seen in FIG. 25, tray retainer (1110) and tissue receiving trays (1030) are initially separated from each other. Optionally, tray retainer (1110) may also be separated from probe (100) at this stage. At this stage, tray retainer (1110) and tissue receiving trays (1130) are manipulated relative to each other to insert tissue receiving trays (1130) into tray retainer (1110). Specifically, fastener (1138) of each tissue receiving tray (1130) is inserted into a corresponding tray receiving feature (1116) and each tissue receiving tray (1130) is slide axially onto tray retainer (1110).

After tissue receiving trays (1130) are secured to tray retainer (1110), the combination of tray retainer (1110) and tissue receiving trays (1130) may be secured to probe (100). Specifically, tray retainer (1110) is manipulated to engage threaded portion (1144) of translation drive (1140) with translator (1150) of translation drive (1140). Translator (1150) is then rotated relative to probe (100) and tray retainer (1110) to further engage threaded portion (1144) with the corresponding threaded portion of translator (1150).

Once tissue sample holder (1100) is secured to probe (100), it may be desirable to seal tissue receiving trays (1130) relative to probe (100). Such sealing is achieved by further rotating translator (1150) to translate tray retainer (1110) distally, thereby driving the distal end of tissue receiving trays (1130) into a sealing feature of probe (100) such as sealing member (170), seal (602), or other sealing features described herein. Optionally, translator (1150) and/or probe (100) may include indicia or other features to indicate to an operator when a sealing position is reached for translator (1150).

Alternatively, prior to sealing, tissue sample holder (1100) is rotated relative to probe (100) via rotation feature of probe (100) such as grasping feature (184). Rotation of tissue sample holder (1100) at this stage may be desirable to initially index a particular tissue sample chamber (1136) with cutter (150) of probe (100). After such indexing, tissue receiving trays (1130) may be sealed by rotating translator (1150) further as described above.

At one or more stages during a biopsy procedure, it may be desirable to rotate tissue sample holder (1100) after tissue receiving trays (1130) are sealed as described above. Prior to such rotation, it may be desirable to release the seal between tissue receiving trays (1130) and probe (100). To release such a seal, translator (1150) is rotated in an opposite direction. Such opposite rotation drives tray retainer (1110) in a proximal direction via threaded portion (1144). The amount of rotation of translator (1150) at this stage is a degree of rotation sufficient to release the seal, while being insufficient to fully decouple tissue sample holder (1100) from probe (100). Tissue sample holder (1100) may then be rotated via one or more features of probe (100).

### V. Exemplary Alternative Seal Configuration

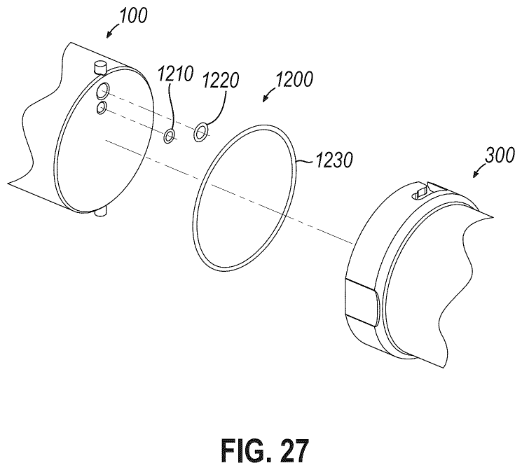

FIG. 27 shows an exemplary alternative sealing feature (1200) that may be readily incorporated into probe (100) described above in-lieu of sealing member (170) and seal (602). Sealing feature (1200) generally includes a plurality of o-rings (1210, 1220, 1230) oriented around one or more fluid junctions of probe (100). For instance, in the present example, probe includes ports for vacuum communication and tissue sample communication. Thus, sealing feature of the present example includes a vacuum o-ring (1210) and a tissue o-ring (1220) to seal the particular areas where fluid is communicated. Additionally, sealing feature (1200) optionally includes an outer perimeter o-ring (1230) to prevent fluid egress from outer areas of the tissue sample holder. In some examples, such outer areas may correspond to a region proximate transparent cover (302) or similar features. As a result of this configuration, the sealing surface in contact with structures such as manifold (310, 710, 810, 910) and/or tissue receiving trays (330, 1030, 1030) may be substantially reduced relative to a sealing feature covering the entire proximal end of probe (100). Consequently, the friction between sealing feature (1200) and structures such as manifold (310, 710, 810, 910) and/or tissue receiving trays (330, 1030, 1030) may be substantially reduced.

### VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy device comprising: a body; a needle extending distally from the body, wherein the needle includes a lateral aperture; a hollow cutter, the cutter being movable relative to the needle to sever a tissue sample, the hollow cutter defining a cutter lumen; a tissue sample holder, wherein the tissue sample holder includes: a rotatable member, and one or more tissue receiving trays, the rotatable member being configured to receive each tissue receiving tray of the one or more tissue receiving trays, the rotatable member being further configured to rotate relative to the cutter to index each tissue receiving tray of the one or more tissue receiving trays relative to the cutter; and a drive mechanism, the drive mechanism including a drive screw, the drive screw being configured to drive both rotation and translation of the rotatable member of the tissue sample holder.

### Example 2

The biopsy device of Example 1, the rotatable member including a threaded receiver configured to engage a threaded portion of the drive screw.

### Example 3

The biopsy device of Example 2, engagement between the threaded receiver of the rotatable member and the threaded portion of the drive screw being configured to drive translation of the rotatable member using rotation of the drive screw.

### Example 4

The biopsy device of any of Examples 1 through 3, the drive mechanism further including a rotation stop, the rotation stop being configured to releasably fix the rotatable member in a fixed rotational position relative to the drive screw.

### Example 5

The biopsy device of Example 4, the rotation stop including a plurality of detents.

### Example 6

The biopsy device of Example 5, each detent of the plurality of detents being disposed on a portion of the rotatable member.

### Example 7

The biopsy device of Example 5, each detent of the plurality of detents extending from an outer surface of the rotatable member.

### Example 8

The biopsy device of Example 5, the rotation stop further including a hard stop disposed on the drive screw and the rotatable member, respectively.

### Example 9

The biopsy device of Example 8, the hard stop of the drive screw and the hard stop of the rotatable member being configured to engage each other to transfer rotation of the drive screw to rotation of the rotatable member.

### Example 10

The biopsy device of Example 4, the rotation stop including a control element, the control element being configured to engage a portion of the rotatable member to releasably hold the rotatable member in a fixed rotational position relative to the drive screw.

### Example 11

The biopsy device of Example 10, the control element defining a plurality of interconnected cam surfaces and stop faces, each stop surface and cam surface being configured to engage a portion of the rotatable member.

### Example 12

The biopsy device of Example 11, the plurality of interconnected cam surfaces and stop faces being arranged to alternate between each cam surface and each stop face.

### Example 13

The biopsy device of Example 11, the plurality of interconnected cam surfaces and stop faces being arranged in an annular shape.

### Example 14

The biopsy device of Example 10, the control element including one or more lock protrusions, the one or more lock protrusions being configured to hold the control element in a fixed position relative to the body.

### Example 15

The biopsy device of any of Examples 1 through 14, the rotatable member including a plurality of passages, each passage being configured to receive at least a portion of a corresponding tissue receiving tray.

### Example 16

A biopsy device comprising: a body; a needle extending distally from the body, wherein the needle includes a lateral aperture; a hollow cutter, the cutter being movable relative to the needle to sever a tissue sample, the hollow cutter defining a cutter lumen; a tissue sample holder, wherein the tissue sample holder includes: a rotatable member, and one or more tissue receiving trays, the rotatable member being configured to receive each tissue receiving tray of the one or more tissue receiving trays, the rotatable member being further configured to rotate relative to the cutter to index each tissue receiving tray of the one or more tissue receiving trays relative to the cutter; and a drive mechanism, the drive mechanism including a rotation drive and a translation drive, the rotation drive being configured to rotate the rotatable member, the translation drive being configured to translate the rotatable member independently of the rotation drive.

### Example 17

The biopsy device of Example 16, the translation drive including a barrel cam, the barrel cam being in communication with the rotatable member to drive translation of the rotatable member via translation of the barrel cam.

### Example 18

The biopsy device of Example 17, the barrel cam defining a cam path, the cam path being configured to receive a cam follower, the cam follower being configured to drive translation of the barrel cam in response to rotation of the barrel cam.

### Example 19

The biopsy device of Example 16, the translation drive including a threaded portion, the threaded portion being configured to engage a portion of the body to convert rotation of a portion of the rotation drive into translation of the rotatable member.

### Example 20

The biopsy device of Example 19, the rotatable member including a rotation tab, the rotation tab being configured to rotate the rotatable member independently of the rotation drive, the rotation tab being further configured to drive rotation of the threaded portion relative to the body to translate the rotatable member.

### Example 21

The biopsy device of Example 19, the translation drive further including a translator, the translator being configured to engage the threaded portion, the translator being further configured to move relative to the body to translate the rotatable member via the threaded portion.

### Example 22

The biopsy device of any of Examples 16 through 21, the rotatable member includes one or more tray receiving features, each tray receiving feature being configured to receive a fastener of a corresponding tissue receiving tray of the one or more tissue receiving trays.

### Example 23

The biopsy device of Example 22, the each tray receiving feature defining a keyed channel.

### Example 24

A biopsy device comprising: a body; a needle extending distally from the body, wherein the needle includes a lateral aperture; a hollow cutter, the cutter being movable relative to the needle to sever a tissue sample, the hollow cutter defining a cutter lumen; a tissue sample holder, the tissue sample holder including: a rotatable member including a cylindrical body and a plurality of tray receiving features extending axially though a portion of the cylindrical body, and a plurality of tissue receiving trays, each tissue receiving tray including a fastener, each tray receiving feature of the rotatable member being configured to receive the fastener of a corresponding tissue receiving tray, the rotatable member being configured to rotate relative to the cutter to index each tissue receiving tray of the one or more tissue receiving trays relative to the cutter; and a drive mechanism, the drive mechanism including a translation drive, the translation drive being configured to translate the rotatable member relative to the body.

### Example 25

The biopsy device of any of Examples 1 through 24, the body including a sealing feature, drive mechanism being configured to translate the rotatable member into the sealing feature to seal each tissue receiving tray relative to the body.

### Example 26

The biopsy device of Example 25, the sealing feature including a plurality of o-rings, each o-ring of the plurality of o-rings being aligned with a fluid junction of the body.

### VII. Conclusion

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy device comprising:
(a) a body;
(b) a needle extending distally from the body, wherein the needle includes a lateral aperture;
(c) a hollow cutter, the cutter being movable relative to the needle to sever a tissue sample, the hollow cutter defining a cutter lumen;
(d) a tissue sample holder, wherein the tissue sample holder includes:
(i) a rotatable member, and
(ii) one or more tissue receiving trays, the rotatable member being configured to receive each tissue receiving tray of the one or more tissue receiving trays, the rotatable member being further configured to rotate relative to the cutter to index each tissue receiving tray of the one or more tissue receiving trays relative to the cutter; and
(e) a drive mechanism, the drive mechanism including a drive screw, the drive screw being configured to drive both rotation and translation of the rotatable member of the tissue sample holder.

2. The biopsy device of claim 1, the rotatable member including a threaded receiver configured to engage a threaded portion of the drive screw.

3. The biopsy device of claim 2, engagement between the threaded receiver of the rotatable member and the threaded portion of the drive screw being configured to drive translation of the rotatable member using rotation of the drive screw.

4. The biopsy device of claim 1, the drive mechanism further including a rotation stop, the rotation stop being configured to releasably fix the rotatable member in a fixed rotational position relative to the drive screw.

5. The biopsy device of claim 4, the rotation stop including a plurality of detents.

6. The biopsy device of claim 5, each detent of the plurality of detents being disposed on a portion of the rotatable member.

7. The biopsy device of claim 5, each detent of the plurality of detents extending from an outer surface of the rotatable member.

8. The biopsy device of claim 5, the rotation stop further including a hard stop disposed on the drive screw and the rotatable member, respectively.

9. The biopsy device of claim 8, the hard stop of the drive screw and the hard stop of the rotatable member being configured to engage each other to transfer rotation of the drive screw to rotation of the rotatable member.

10. The biopsy device of claim 4, the rotation stop including a control element, the control element being configured to engage a portion of the rotatable member to releasably hold the rotatable member in a fixed rotational position relative to the drive screw.

11. The biopsy device of claim 10, the control element defining a plurality of interconnected cam surfaces and stop faces, each stop surface and cam surface being configured to engage a portion of the rotatable member.

12. The biopsy device of claim 11, the plurality of interconnected cam surfaces and stop faces being arranged to alternate between each cam surface and each stop face.

13. The biopsy device of claim 11, the plurality of interconnected cam surfaces and stop faces being arranged in an annular shape.

14. The biopsy device of claim 10, the control element including one or more lock protrusions, the one or more lock protrusions being configured to hold the control element in a fixed position relative to the body.

15. The biopsy device of claim 1, the rotatable member including a plurality of passages, each passage being configured to receive at least a portion of a corresponding tissue receiving tray.

16. A biopsy device comprising:
(a) a body;
(b) a needle extending distally from the body, wherein the needle includes a lateral aperture;
(c) a hollow cutter, the cutter being movable relative to the needle to sever a tissue sample, the hollow cutter defining a cutter lumen;
(d) a tissue sample holder, wherein the tissue sample holder includes:
(i) a rotatable member, and
(ii) one or more tissue receiving trays, the rotatable member being configured to receive each tissue receiving tray of the one or more tissue receiving trays, the rotatable member being further configured to rotate relative to the cutter to index each tissue receiving tray of the one or more tissue receiving trays relative to the cutter; and
(e) a drive mechanism, the drive mechanism including a rotation drive and a translation drive, the rotation drive being configured to rotate the rotatable member, the translation drive being configured to translate the rotatable member independently of the rotation drive.

17. The biopsy device of claim 16, the translation drive including a barrel cam, the barrel cam being in communication with the rotatable member to drive translation of the rotatable member via translation of the barrel cam.

18. The biopsy device of claim 17, the barrel cam defining a cam path, the cam path being configured to receive a cam follower, the cam follower being configured to drive translation of the barrel cam in response to rotation of the barrel cam.

19. The biopsy device of claim 16, the translation drive including a threaded portion, the threaded portion being configured to engage a portion of the body to convert rotation of a portion of the rotation drive into translation of the rotatable member.

20.-23. (canceled)

24. A biopsy device comprising:
(a) a body;
(b) a needle extending distally from the body, wherein the needle includes a lateral aperture;
(c) a hollow cutter, the cutter being movable relative to the needle to sever a tissue sample, the hollow cutter defining a cutter lumen;
(d) a tissue sample holder, the tissue sample holder including:
(i) a rotatable member including a cylindrical body and a plurality of tray receiving features extending axially though a portion of the cylindrical body, and
(ii) a plurality of tissue receiving trays, each tissue receiving tray including a fastener, each tray receiving feature of the rotatable member being configured to receive the fastener of a corresponding tissue receiving tray, the rotatable member being configured to rotate relative to the cutter to index each tissue receiving tray of the one or more tissue receiving trays relative to the cutter; and
(e) a drive mechanism, the drive mechanism including a translation drive, the translation drive being configured to translate the rotatable member relative to the body.

25. (canceled)

26. (canceled)
